(19) United States
(12) Patent Application Publication
(10) Pub. No.: US 2007/0010429 A1
(43) Pub. Date: Jan. 11, 2007

(54) Phospholipid formulations and uses thereof in lung disease detection and treatment

(75) Inventors: Martin Post, Toronto (CA); Ross Ridsdale, Milford, OH (US); Matthias Roth-Kleiner, Epalinges (CH)

Correspondence Address: JAGTIANI + GUTTAG, 10363-A DEMOCRACY LANE, FAIRFAX, VA, 22030, US

(73) Assignee: The Hospital For Sick Children, Toronto (CA)

(21) Appl. No.: 11/412,975

(22) Filed: Apr. 28, 2006

Related U.S. Application Data
(60) Provisional application No. 60/676,949, filed on May 3, 2005.

(51) Int. Cl. A61K 38/17; G01N 33/92; A61K 31/685

(52) U.S. Cl. 514/7; 514/78; 436/71

(57) ABSTRACT

Disclosed are methods and compositions that are useful in the detection and therapy of diseases (e.g., emphysema) and damage that afflict the lungs. In some aspects, the compositions comprise a formulation enriched for a species of phosphatidylcholine, such as palmitoylmyristoyl phosphatidylcholine (16:0/14:0PC). The compositions may further be described as lung surfactant supplement preparations particularly useful in the treatment of pulmonary diseases and afflictions prevalent among premature infants, and in particular, Respiratory Distress Syndrome (RDS). A PC marker is also disclosed, 16:0/14:0PC, that may be used to detect pulmonary disease or reduced/compromised alveolar function in an animal. Phospholipid profiles of 16:0/14:0PC, 16:0/16:1PC and 16:0/16:0PC are also provided, and are correlated with particular pulmonary diseased states.

### CROSS-REFERENCE TO RELATED APPLICATIONS

This application makes reference to Provisional U.S. Patent Application Ser. No. 06/676,949, filed May 3, 2005. The entire disclosure and contents of the above application is hereby incorporated by reference.

### BACKGROUND

1. Field of the Invention

The present invention relates generally to the field of surfactant preparations and surfactant supplements for the lung. The invention also relates to the field of lung disease detection and treatment, as a marker of lung disease comprising a characteristic phospholipid profile is presented, and is correlated with specific developmental and disease-related changes in lung tissue.

2. Related Art

Pulmonary surfactant is a complex mixture of lipids and proteins that is synthesized and secreted by alveolar type II epithelial cells. These cells secrete this mixture of lipids and proteins into the thin liquid layer that lines the epithelium. Once in the extracellular space, surfactant reduces surface tension at the air-liquid interface of the lung, a function that requires an appropriate mix of surfactant lipids and the hydrophobic proteins, surfactant protein (SP)-B and SP-C (1, 2). Of the surfactant lipids, 80-90% are phospholipids, with the rest being neutral lipids. The most abundant phospholipid species is phosphatidylcholine (PC) with dipalmitoyl-PC (16:0/16:0PC) being important in attaining acceptable surface tensions (near 0 mN/m (3-7)). Although saturated palmitoylmyristoyl-PC (16:0/14:0PC) and monounsaturated palmitoylpalmitoleoyl-PC (16:0/16:1PC) are also prevalent in most mammalian lung surfactants (7), their contribution to surfactant function is not well understood. In vitro studies have demonstrated that dipalmitoyl-PC (16:0/16:0PC) does not spread well at the air/liquid interface (8, 9). One possibility is that palmitoylmyristoyl-PC (16:0/14:0PC) and palmitoylpalmitoleoyl-PC (16:0/16:1PC) assist in the surface spreading of dipalmitoyl-PC (16:0/16:0PC) (10, 11).

The phospholipids palmitoylmyristoyl-PC (16:0/14:0PC) and palmitoylpalmitoleoyl-PC (16:0/16:1PC) may contribute to dynamic surfactant functions during mammalian respiration (4). Fractional concentrations of both of these PC species in lung surfactant have been found to correlate with respiratory rates in mammals (4). With increasing respiratory rates, both palmitoylmyristoyl-PC (16:0/14:0PC) and palmitoylpalmitoleoyl-PC (16:0/16:1PC) concentrations in surfactant are increased. Thus, the fractional concentrations of palmitoylmyristoyl-PC (16:0/14:0PC) and palmitoylpalmitoleoyl-PC (16:0/16:1PC) in surfactant are adapted to the physiological needs of the mammalian lung (4).

There are two events during lung development that may have specific surfactant needs. First, at birth, the lungs require large amounts of surfactant to convert the fluid-filled airspaces into gas-exchange units with a stable air/liquid interface. Failure to establish a low surface tension air/liquid interface at the distal airspace results in respiratory distress syndrome (RDS), a relatively common complication of premature birth as a result of delayed lung fluid clearance and/or pulmonary surfactant insufficiency (12, 13). Second, in several mammalian species distal lung development proceeds postnatally, such that the alveoli are formed exclusively after birth (e.g. rats and mice) or predominantly after birth (e.g. humans).

The process of alveolarization involves the division of the preexisting voluminous terminal air sacs (saccules) into smaller units, the alveoli, by secondary septa. These septa grow out from the saccular walls into the air spaces in a centripetal manner. As a result there is an increase in the number of terminal gas exchange units (14). Although these newly formed alveoli have less volume there is a substantial net increase in total surface area (14). In rats (14) and mice (15), the bulk of secondary septation takes place between postnatal days 4 and 14. Human alveolarization occurs mainly between 36 weeks of gestation and 18 months of age (16). The completion of alveolarization results in an increased number of terminal airway units which continue to grow in size to further expand surface area into adult life. Whether these morphological surface changes during alveolarization impact on the composition of functionally important surfactant PC species is also unknown.

The state of the art demonstrates that a medical need continues to exist for preparations that are effective and useful for enhancing assessment of lung development and alveolarization, especially in monitoring and treating premature infants. In addition, a need continues to exist for a reliable marker for assessing lung disease and lung developmental stages, particularly as related to alveolarization, such that an appropriate therapeutic preparation, such as a surfactant preparation and/or other therapeutic intervention may be made available to the patient.

### SUMMARY

The present invention in an overall and general sense relates to the characterization and discovery of particularly defined phospholipid preparations having significance in the identity of pathological and developmental abnormalities in the lung, and particularly to the health and development of alveolar tissue in the lung.

The present invention provides a variety of pharmaceutically acceptable preparations of specific formulations of phospholipid PC preparations. These preparations may be formulated for delivery to a patient in need thereof according to a variety of techniques well known to those of skill in the formulary and pharmaceutical arts.

The invention also provides a reliable marker for alveolar pathology, and as such provides a clinical indicator of reduced alveolarization or pulmonary disease state in a subject animal. This marker comprises a particular phospholipid, palmitoylmyristoyl-PC (16:0/14:0PC). The relative concentration of this phospholipid species in a subject animal sample, such as a lung sample, has been correlated by the present inventors with an increased incidence of a variety of pulmonary diseases and conditions of compromised pulmonary function in infants and adults. In particular embodiments, a reduced relative concentration of palmitoylmyristoyl-PC (16:0/14:0PC) in a subject animal sample, in particular a lung sample, relative to a control animal sample concentration of palmitoylmyristoyl-PC (16:0/14:0PC), is diagnostic of reduced alveolarization of the lung, possibly related to pulmonary disease or exposure to a toxic substance. In particular embodiments of the method, the concentration of palmitoylmyristoyl-PC (16:0/14:0PC) is reduced at least 20%, 25%, 40%, or as much as 90% to 100%, relative to a control sample palmitoylmyristoyl-PC (16:0/14:0PC) concentration, in animals having pulmonary disease or having been exposed to a toxic solid or air-born chemical or pollutant.

By way of example, representative lung diseases and pathologies for which the presently disclosed marker and preparations may be used to identify and treat include, but are not limited to, emphysema, respiratory distress syndrome (both adult and infant), idiopathic pulmonary fibrosis, bronchopulmonary dysplasia (chronic lung disease), asthma, and congenital malformations (eg. lung hypoplasia, congenital lobar emphysema, congenital cystic adenomatoid malformations, congenital alveolar capillary dysplasia, alphal antitrypsin deficiency and others), lung ischemia-reperfusion injury (LIRI), chronic lung disease (CLD) and meconium aspiration syndrome (MAS). The marker may also be used to predict risk for development of more advanced forms of pulmonary distress/damage. For example, a correlation is known to exist between relative 16:0/14:0 concentration of an infant sample lung sample and to a heightened incidence for the development of respiratory distress. This particular prognostic indicator for predicting risk of progressive lung disease finds clinical application for use in a method for assessing risk for developing more serious or continued pulmonary disease in an infant having respiratory distress. A heightened risk of developing progressively more severe pulmonary distress exists particularly in an infant maintained or having been maintained on a pulmonary respirator or other assisted breathing apparatus.

A diagnostic PC profile has also been identified and correlated with a diseased or compromised pulmonary state in an animal resulting from pulmonary disease or exposure to a toxic substance or environmentally compromising event (high O₂, extreme pressure changes, etc.). In particular embodiments, the PC profile comprises the animal's relative lung sample palmitoylmyristoyl-PC(16:0/14:0PC) concentration. An animal lung sample having a sample palmitoylmyristoyl-PC (16:0/14:0PC) less than a level of palmitoylmyristoyl-PC(16:0/14:0PC) in a control animal lung sample is diagnostic of a diseased pulmonary state, a reduced or compromised alveolarization state in the animal, or of exposure to a pulmonary toxic substance. Such a diagnostic use may find application in settings where humans have been or are exposed to potentially compromising inhaled substances. Examples of such potentially compromising inhaled substances include by way of example, and not limitation, air-born industrial and environmental chemicals (smog), smoke, inhaled steroids (such as particular inhaled asthma medicaments, “puffers”), dexamethasone, chemical waste products, alcohols, and the like.

In some embodiments, the PC profile characteristic of pulmonary exposure to a toxic or potentially toxic substance, such as dexamethasone, comprises a measure of the total phospholipid PC content of a subject animal lung sample. A PC profile of this nature in an animal having been exposed to a toxic substance would be elevated relative to the total PC content of a control animal lung sample. The total PC content may be further defined as comprising a measure of the lung sample palmitoylpalmitoleoyl-PC (16:0/16:1PC) and palmitoyloleoyl-PC (16:0/18:1PC) content. The total PC measure in an animal having been exposed to a toxic substance will be elevated about 10% to about 20%, or more, over total PC content observed from lung tissue of a control animal. In particular embodiments, the relative total PC content/concentration may be used as part of a method for detecting pulmonary damage or reduced pulmonary function resulting from toxin or chemical exposure, such as from exposure to dexamethasone.

In other embodiments, the total PC content of an animal lung sample may be used as part of a method to detect or diagnose exposure to high O₂ concentrations. In these embodiments, the total PC content comprises a measure of palmitoylpalmitoleoyl-PC (16:0/16:1PC) and palmitoyloleoyl-PC (16:0/18:1PC). The total PC content will be reduced in a subject animal that had been exposed to O₂, relative to a control animal lung sample total PC content/concentration. The amount/concentration of total PC in an O₂ exposed animal lung sample will be reduced 20%, 40%, or even more, compared to a control animal lung sample.

In other embodiments, the PC profile in a diseased or pulmonary compromised animal may be detected through a measure of the dipalmitoyl-PC (16:0/16:0PC) concentration of the animal lung sample. In this context, an elevated (about 20% to about 40%) concentration of dipalmitoyl-PC (16:0/16:0PC) compared to a control animal lung sample, is diagnostic of diseased or compromised pulmonary function.

A diagnostic PC alveolar content profile is specifically defined for animals having reduced alveolar function as a result of disease, such as in emphysema, respiratory distress syndrome (infant and adult), and other disease states related to lung and pulmonary function. This profile includes a reduced relative concentration of lung tissue palmitoylmyristoyl-PC (16:0/14:0PC). A method employing this profile to diagnose and detect disease is provided. In one aspect, the method comprises obtaining a lung sample from a subject animal to provide a subject animal lung sample, measuring the amount of palmitoylmyristoyl-PC (16:0/14:0PC) in the subject animal lung sample, and comparing the amount of palmitoylmyristoyl-PC (16:0/14:0PC) in the subject animal lung sample to an amount of palmitoylmyristoyl-PC(16:0/14:0PC) in a control animal lung sample, wherein a reduced concentration of palmitoylmyristoyl-PC (16:0/14:0PC) in the subject animal lung sample relative to the concentration of palmitoylmyristoyl-PC (16:0/14:0PC) in the control animal lung sample is diagnostic of reduced alveolar function/architecture or alveolar damage attendant disease.

The animal samples that may be analyzed and used for the various diagnostic, therapeutic and forensic applications described herein may constitute an infant, fetal, adult, or even cadaver harvested lung tissue specimen. The preparations, markers, and methods of the invention are suitable for both human and veterinary use, and therefore finds application for use in humans, domestic animals (horses, cats, dogs, pigs), and other commercially valuable animal species (monkeys, lambs, , rats, mice, hamsters, guinea pigs, bears, deer, cows, chickens, etc.).

The present invention also provides a number of surfactant and surfactant supplement preparations tailored to treat and manage lung disease and compromised alveolar/pulmonary function in a newborn/infant (to 18 months). These particular surfactant and surfactant supplements of the invention employ the correlation established by the present inventors between the absolute and fractional changes in concentrations of functionally important PC species (dipalmitoyl-PC (16:0/16:0PC), palmitoylmyristoyl-PC (16:0/14:0PC) and palmitoylpalmitoleoyl-PC(16:0/16:1PC) ) immediately after birth. For example, several unique developmental stage dependent PC profiles are identified here, and used to formulate custom tailored phospholipid surfactant preparations and surfactant supplements that will enhance postnatal lung development and deter/inhibit damage to alveolar tissue.

The specific compositional amount of each of these 16:0/16:1PC, 16:0/14:0PC and 16:0/16:0PC species that will be in each formulation will vary with the specific developmental stage and pathology of the subject being treated. In some embodiments, the specific composition of the PC formulations will be enriched for the PC species identified to be deficient in a subject animal. One such surfactant PC formulation tailored for use in infants with surfactant deficiency is enriched for palmitoylmyristoyl-PC (16:0/14:0PC). By way of example, such a formulation would include a concentration of about 20% to about 50% fractional concentration of palmitoylmyristoyl-PC (16:0/14:0PC). Another such surfactant PC formulation may be enriched for palmitoylpalmitoleoyl-PC (16:0/16:1PC) for use in newborns immediately or shortly after birth to enhance surfactant spreading capacities. By way of example, such a formulation would include a concentration of about 20% to 40% fractional concentration of palmitoylpalmitoleoyl-PC (16:0/16:1PC). By way of reference, conventional surfactant preparations, such as Curosurf®, contain 80 mg/ml phospholipids, of which 70% are phosphatidylcholine. About 30% of the phosphatidylcholine should be palmitoylpalmitoleoyl-PC (16:0/16:1PC) which is about 15-20 mg/ml. An average treatment dosage of Curosurf® administered to a newborn infant is about 100-200 mg/kg per dose.

The surfactants and surfactant supplements may also be used as a carrier for therapeutically, prophylactically, and/or diagnostically suitable or active substance or substances, e.g., pulmonary drug delivery.

The invention also provides a pharmaceutical kit. In some embodiments, the kit comprises a container means comprising a preparation enriched for 16:0/14:0PC, 16:0/16:1PC, 16:0/18:0PC, 16:0/18:1PC, or a particular desired mixture thereof, and a second container means comprising a suitable pharmaceutical grade carrier solution. This carrier solution will be suitable for suspending or dissolving the contents of the first container means to provide a preparation of the desired concentration in a ready to use form for the subject patient. It is envisioned that for use in an infant having RDS, the preparation will be enriched for 16:0/14:0PC. The kit may optionally also include an instructional sheet. This instructional sheet may include instructions on how the PC is to be reconstituted depending upon the particular use for which it will be made, directions for administration, recommended dosages, storing conditions, and appropriate warnings. The kit may also include a device for facilitating administration of the preparation to the subject, such as a tracheal tube, aspirator, or other appropriate device.

The surfactants and surfactant supplements of the invention may also include surfactant proteins, such as SP-A, SP-B, SP-C, SP-D, or mixtures thereof. The palmitoylmyristoyl-PC (16:0/14:0PC), 16:0/16:1PC, 16:0/16:0PC, 16:0/18:0PC and 16:0/18:1PC of the surfactants and surfactant supplements may be of synthetic origin and obtained from other than a porcine or bovine tissue source. Some embodiments of the surfactants and surfactant supplements may be prepared from phospholipid species obtained or derived from porcine or bovine tissue origin, or obtained from recombinant cells engineered to express the appropriate desired ingredient.

In particular embodiments, the surfactant and surfactant supplements are formulated so as to be suitable for delivery through a tracheal tube into the lungs of a patient subject animal. In other embodiments, the formulation may be prepared so as to be suitable for delivery as an aerosol. These and other delivery forms are readily prepared for use in the practice of the present invention given the specific types and ratios of specific phospholipids described herein, and those formulation techniques known to those in the formulary arts, such as are described in Remington's Pharmaceutical Sciences (61), which text is specifically incorporated herein by reference.

The following abbreviations are used through out the description of the invention:
16:0/14:0PC—Palmitoylmyristoyl Phosphatidylcholine;
16:0/16:0PC—Dipalmitoyl Phosphatidylcholine;
16:0/16:1PC—Palmitoylpalmitoleoyl Phosphatidylcholine;
16:0/18:1PC—Palmitoyloleoyl Phosphatidylcholine;
ALI—Acute Lung Injury;
ARDS—Adult Respiratory Distress Syndrome;
BALF—Bronchoalveolar Lavage Fluid;
BPD—Broncho pulmonary Dysplasia;
CLD—Chronic Lung Disease;
COPD—Chronic Obstructive Pulmonary Disease;
DPPC—Dipalmitoyl Phosphatidylcholine (16:0/16:0PC) (also known as colfosceril palmitate);
EMO—Extracorporeal Membrane Oxygenation;
IPF—Idiopathic Pulmonary Fibrosis;
IRDS—Infant Respiratory Distress Syndrome;
LIRI—Lung Ischemia-Reperfusion Injury;
MAS—Meconium Aspiration Syndrome;
PC—Phosphatidylcholine;
RDS—Respiratory Distress Syndrome;
SP—Surfactant Protein.

### BRIEF DESCRIPTION OF THE DRAWINGS

The invention will be described in conjunction with the accompanying drawings, in which:

FIGS. 1(a)-1(c): Developmental profile of total PC: (1a) Total PC content in bronchoalveolar lavage fluid (BALF) of untreated rats. (1b) Total PC content of BALF during late fetal gestation. (1c) Total PC content of BALF during first 4 days after birth. Mean±SE, n=4 animals per time point [except for samples taken in the first 20 minutes postpartum (n=3 animals) and day 10 (n=8 animals)]. * P<0.01.

FIGS. 2(a)-2(d): Concentration of three major PC species in bronchoalveolar lavage fluid during rat development: (2a) dipalmitoyl-PC (16:0/16:0PC), (2b) palmitoylpalmitoleolyl-PC (16:0/16:1PC), (2c) palmitoylmyristoyl-PC (16:0/14:0PC). (2d) The three individual PC species presented as percent of total PC. Mean±SE, n=4 animals per time point [except for samples taken in the first 20 minutes postpartum (n=3 animals) and day 10 (n=8 animals)].

FIGS. 3(a)-3(c): Percentage changes of the three major PC species in bronchoalveolar lavage fluid in mice and rats at late fetal gestation (mouse E18 (embryonic day 18), rat E22), at peak of alveolarization (postnatal day (PN) day 10, mice; PN14, rat), and mature juveniles (PN22). Mice (white bars) and rats (black bars). (3a) dipalmitoyl-PC (16:0/16:0PC), (3b) palmitoylpalmitoleolyl-PC (16:0/16:1PC), (3c) palmitoylmyristoyl-PC (16:0/14:0PC). Individual species are presented as percent of total PC. Mean±SE, n=4 animals per time point [except for mice sample E18 (n=10) and PN 22 (n=10)]. * P<0.01.

FIGS. 4(a)-4(h): Bronchoalveolar lavage content of total PC (4a, 4e), dipalmitoyl-PC (16:0/16:0PC) (4b, 4f), palmitoylpalmitoleolyl-PC (16:0/16:1PC) (4c, 4g), palmitoylmyristoyl-PC (16:0/14:0PC) (4d, 4h). Panels a-d from 10 day old rats. White bars: control rats; grey bars: rats exposed to 60% O₂ and black bars: dexamethasone treated rats. Panels 4e-4h from 4 to 14 day old rats. Solid line: control rats and dashed line: rats exposed to 60% O₂. Mean±SE, n=4 animals per treatment and time point. * P<0.01.

FIGS. 5(a)-5(b): Bronchoalveolar lavage and tracheal aspirate content of (5a) dipalmitoyl-PC (16:0/16:0PC), (5b) palmitoylmyristoyl-PC (16:0/14:0PC) from infants and adults with and without alveolar pathology. Mean±SE, RDS, n=19; CLD, n=8; lung tumor, n=8; transplant, n=4; COPD, n=3; IPF, n=3. * P<0.01.

FIGS. 6(a)-6(c): Correlations between palmitoylmyristoyl-PC (16:0/14:0PC) and alveolar curvature. (6a) Developmental changes of mean alveolar radius and palmitoylmyristoyl-PC (16:0/14:0PC) concentration in bronchoalveolar lavage. Alveolar radii were calculated from published data of Burri et al.(14), divided in half (open circles), and from Blanco et al. (55), for day 2, 23, 40, and Blanco et al., (54) for day 14, 60, considering the form of alveoli as a sphere (Radius=3_{rd} root of [3× mean alveolar volume/4π]) (full circles, solid line). BALF concentration of palmitoylmyristoyl-PC (16:0/14:0PC) are shown as percent of total PC. (6b) Light scattering of spontaneously formed liposomes from BALF lipids. A correlation plot of the percentage of palmitoylmyristoyl-PC (16:0/14:0PC) in the BALF versus the radius of spontaneously formed liposomes is shown. Mean±SE, n=4 separate samples. (6c) Developmental changes of dipalmitoyl-PC (16:0/16:0PC) (solid line) and palmitoylmyristoyl-PC (16:0/14:0PC) (dashed line) content in alveolar type II epithelial cells.

FIG. 7: Percent of 16:0/18:1 obtained from a normal rat profile. Developmental profile of palmitoyloleoyl-PC (16:0/18:1PC) concentration in BALF samples from rat lung.

FIG. 8 Comparison of commercially available surfactant compositions, Survanta®, Curosurf®, and BLES. Phospatidylcholine concentrations in 3 natural surfactants (Survanta®, Curosurf® and BLES) preparations and tracheal aspirate samples from 3-day newborn human (3 days postpartum) (white bar) and 35—week gestation (solid black bar) human infants.

FIG. 9: Prognostics indications of tracheal aspirate 16:0/14:0PC concentrations in human infants for development of bronchopulmonary dysplasia (or chronic lung disease). A—Samples taken from patients diagnosed with RDS that did not develop BPD (CLD); B—Samples taken from patients diagnosed with BPD; and C—Samples taken from patients diagnosed with RDS that did not develop BPD (CLD).

### DETAILED DESCRIPTION

It is advantageous to define several terms before describing the invention. It should be appreciated that the following definitions are used throughout this application.

For administration by inhalation, compounds of the present invention can be delivered in the form of aerosol spray presentation from pressurized packs or a nebulizer, with the use of a suitable propellant. In the case of a pressurized aerosol, the dosage unit can be determined by providing a valve to deliver a metered amount. The formulation would be prepared as a powder for administration by inhalation. A powder form is obtained, for example, by mixing liquid lung surfactant preparations, for example aqueous suspensions, with aqueous suspensions of an enriched concentration of the palmitoylpalmitoleoyl-PC (16:0/16:1PC) and/or palmitoylmyristoyl-PC (16:0/14:0PC) and/or dipalmitoyl-PC (16:0/16:0PC), alone or in combination with other desired ingredients, and then subject to drying procedures whereby the liquid component is removed. Administration by inhalation can also be carried out by atomizing solutions or suspensions which contain the compositions according to the invention.

The compositions according to the invention may also be formulated in a liquid form for intratracheal or intrabronchial administration, or for use as a lung wash/lavage fluid.

All of the formulations and additives of the invention may be prepared by procedures familiar to those skilled in the art, if appropriate using further suitable pharmaceutical auxiliaries. Compositions according to the invention advantageously contain the species of PC preparations described herein, and in particular an enriched concentration of the palmitoylpalmitoleoyl-PC (16:0/16:1PC), palmitoylmyristoyl-PC (16:0/14:0PC) and/or dipalmitoyl-PC (16:0/16:0PC), alone or in combination with other desired ingredients, e.g. surfactant proteins.

Definitions

Where the definition of terms departs from the commonly used meaning of the term, applicant intends to utilize the definitions provided below, unless specifically indicated.

It should be noted that the singular forms, “a”, “an” and “the” include reference to the plural unless the context as herein presented clearly indicates otherwise.

A “lung sample” is defined, by way of example, as a lung lavage sample, a lung tissue aspirate, a lung tissue biopsy, a fetal tissue biopsy, a villus tissue sample, a tracheal aspirate, a sputum sample, induced sputum sample, or any other pulmonary derived tissue sample that includes an adequate amount of surfactant or of alveolar or other lung tissue cells or combination of cells sufficient to approximate the relative phospholipid content and/or mixture of phospholipids species content. Hence, the sample should provide sufficient tissue needed to extract the component phospholipids in the sample.

The technique that will typically be used to analyze lipid content in an animal lung sample is by a mass spectral analysis of lipids extracted from the subject lung sample. Yet another technique that may be used to quantify and qualitate phospholipid species in a tissue sample has been described in Bernhard et al., Am. J. Respir. Crit. Care Med. 2004, 170 (1):54-8, which is specifically incorporated herein by reference. This technique may be used to specifically quantify surfactant PC synthesis in vivo. This method employs deuteriated choline coupled with electrospray ionization tandem mass spectrometry. Advantages associated with both mass spectral techniques include the ability to accurately identify and quantitate several different phospholipids at once in a test sample.

The compositions and other biological factors may be administered through any known means. Alveolar administration, such as in an inhailable or aerosol formulation, provides an efficient approach for providing and delivering the preparation to the tissue site in need thereof, such as the lungs.

An “enriched concentration” of a particular, for example, phospholipid species, as described herein is defined as a concentration of the specified phospholipid that is greater than any other phospholipid species in the preparation. For example, where a formulation is described as an “enriched 16:0:14:0PC” formulation, the preparation will include a greater concentration of the phospholipid 16:0/14:0PC, than any other species of phosopholipid in the preparation. By way of example, this particular formulation may include a phospholipid component that comprises at least about 50% 16:0/14:0PC (w/w) of the total phospholipid content of the formulation. An “enriched” phospholipid may comprise between 50% to about 99% of the specified phospholipid, relative to the total phospholipid component of the formulation. This percentage may also vary as between about 60% to about 90%, about 75% to about 98%, about 80% to about 95%, about 85% to about 95%, or even about 98% to 99% (w/w) of the phospholipid species. The “enriched” formulations may also be essentially free of any other phospholipid species other than the species of phospholipid for which it has been enriched. One such embodiment of the present formulations is essentially free of any other phospholipid other than 16:0:14:0PC.

A “therapeutically effective amount” of an active agent or combination of agents as described herein is understood to comprise an amount effective to elicit the desired response, but insufficient to cause a toxic reaction. A desired response, for example, may constitute the formation of a sufficient and/or acceptable alveolar film layer of phospholipids at the desired air/tissue interface. The dosage and duration of treatment of the preparation to be administered to a subject will be determined by the health professional attending the subject in need of treatment, and will consider the age, sex, weight, extent of existing alveolar development and/or damage of the subject, and specific formulation of phospholipids being used as treatment for the subject.

### EXAMPLES

The following non-limiting examples are illustrative of the present invention, and should not be construed to constitute any limitation of the invention as it is described in the claims appended hereto.

### Example 1

### Materials and Methods

The present example describes the experimental protocols used to characterize the 16:0/14:0PC-enriched and other phospholipid preparations described herein. This example also sets forth the protocols that were employed in examining the developmental stages in the lung, and the activity of the present preparations on the specific developmental stages in the lung, especially those during early postnatal life. This example also sets forth the procedures that were used to examine phospholipid profiles in human adult lungs, such as that characteristic of adult human lungs in a patient with emphysema.

Animal Models—

Timed-pregnant female Wistar rats and C57/B16 mice were obtained from Charles River (St. Constant, Qc, Canada). All animal protocols were in accordance with Canadian Counsel of Animal Care guidelines and were approved by the Animal Care and Use Committee of the Hospital for Sick Children. Developmental profile: At fetal day 19 and 22 (term=23 days) the pregnant rat was anesthetized and the pups delivered by cesarean section, while postpartum rodents were removed from their mother directly before sacrifice. Dexamethasone treatment: Newborn Wistar rats were injected daily for 4 days with dexamethasone (Sabex, Boucherville, Quebec, Canada) diluted in isotonic saline starting at postnatal day 1 according to previously described protocols (17). Hyperoxia exposure: The oxygen treatment was performed by housing the rat pups with their mother in chambers containing either 21% or 60% oxygen, starting at day 1 until day of lavage (i.e. postnatal day 4, 7, 10 or 14) (18).

Bronchoalveolar Lavage Fluid Collection—

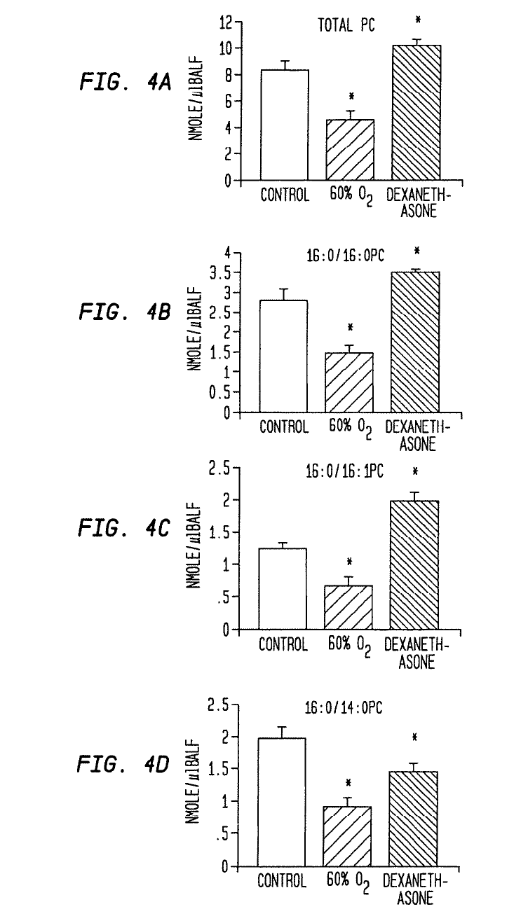

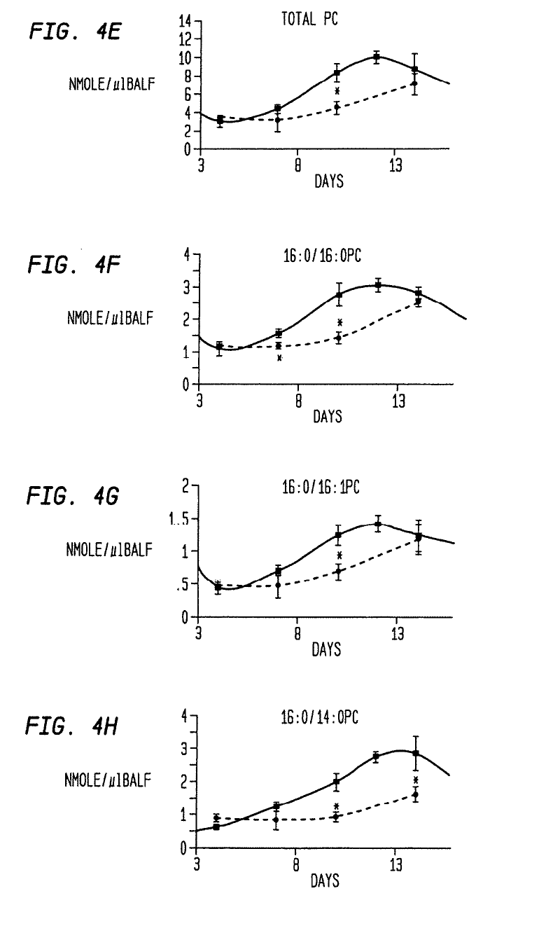

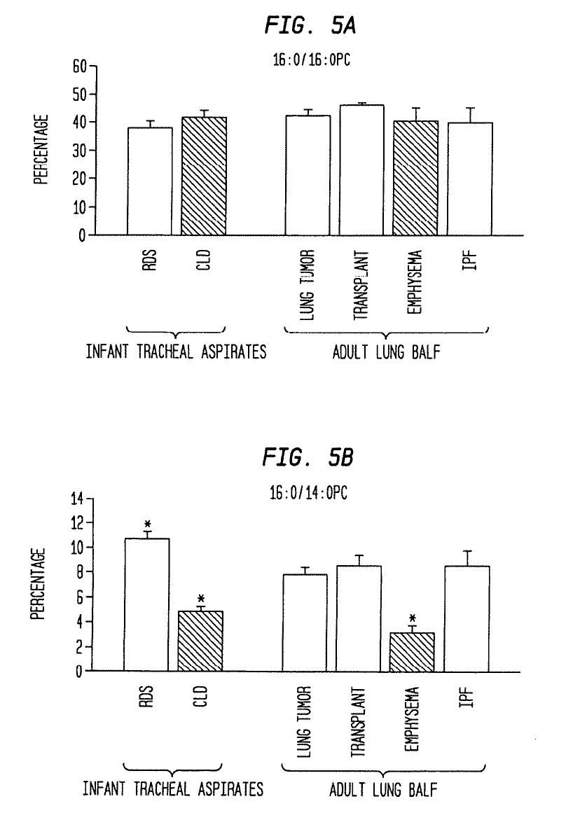

After sacrificing the animals, a needle (blunted tip) was inserted through a tracheostomy and the lungs were lavaged with a buffer composed of phosphate-buffered saline augmented with 0.05 mg/ml of 70 kDa dextran-FITC [Molecular probes, Burlington, ON, Canada]. The inert fluorescent marker was included to determine lavage recovery (see FIG. 4e).

Tracheal Aspirate and Bronchoalveolar Lavage Fluid Collection—

Tracheal aspirates from infants were obtained by irrigation through the endotracheal tube using 1 ml of saline. Respiratory distress syndrome (RDS) was defined as a requirement for exogenous surfactant at the time of birth for babies 27 weeks gestational age or older. For babies below 27 weeks gestational age, RDS was defined as the ongoing need for mechanical ventilation following exogenous surfactant therapy. Although the diagnosis was made at the time of birth, the samples were collected from 29 to 42 weeks corrected gestational age (median: 31 weeks; n=19). Bronchopulmonary dysplasia (BPD) was defined by a consistent clinical course and x-ray changes. This was later confirmed by a continued requirement for supplemental oxygen at 36 weeks corrected gestational age. The samples of BPD patients were collected between 28 and 43 weeks of gestation (median: 32 weeks; n=8). Prenatal steroids (Celestone, Schering, Berlin Germany) were administered to 21 of 28 patients, equally distributed between the RDS and BPD groups. For adult patients bronchoscopy was done using 50 ml of saline for BAL. Samples from emphysematic patients (n=3) and patients with idiopathic pulmonary fibrosis (n=3) were collected prior to lung transplantation.

Control samples were taken prior to transplantation from patients with no alveolar complications (n=8) and from patients with lung tumors (n=4) with little or no chronic obstructive pulmonary disease (COPD). Samples were spun at 1000 g for 5 minutes to remove cellular material. All patient samples were obtained in accordance with Health Canada's Research Ethics Board guidelines.

Mass Spectral Analysis of PC—

BALF samples were spiked with 1 μg of deuterated dipalmitoyl-PC (16:0/16:0PC) (Avanti polar lipids, Alabaster Ala., USA) as an internal standard, and then extracted (19). Lipids were analyzed using an API4000 mass spectrometer [MDS SCIEX, Concord, Ontario, Canada) (20).

Light Scattering—

BALF samples, containing 50 nM of PC, were extracted and lipids were dried under nitrogen. Lipid samples were then reconstituted in 1 ml of saline at 37° C. followed by bath sonication. Following one freeze-thaw cycle the vesicle size was determined by dynamic light scattering (21) using a Malvern Mastersizer X (Malvern Instruments Ltd., Worcestershire, United Kingdom).

Laser Capture Microdissection—

Cryo-embedded lung sections were processed as previously described (20). Alveolar Type II epithelial cells were visualized using a rabbit polyclonal antibody against pro-NSP-C (private source) followed by a FITC-conjugated secondary goat anti-rabbit IgG antibody (Calbiochem, San Diego, Calif., USA). Approximately 200 alveolar Type II epithelial cells were captured using a PixCell II System (Arcturus, Mountain View, Calif., USA), lipids extracted and analyzed by mass spectrometry.

Statistics—

All values are shown as mean±standard error (SE). Statistical analysis was done by Student's t-test or, for comparison of more than two groups, by one-way analysis of variance followed by Duncan's multiple range comparison test, with significance defined as P<0.05.

### Example 2

### Preparation of Phospholipid Surfactant Formulations, Pharmaceutical Preparations and Kits

The present example describes the methods by which the various surfactant preparations and lung surfactant replacement preparations may be formulated for use according to the present invention. However, it is to be understood that other practical and well known formulation techniques known to those of skill in the art may also be used, given the teaching provided herein of the specific types of phospholipids, the phosphatidylcholines and ratios of the phosphatidylcholines that are demonstrated to have particular and specific activity for enhancing pulmonary function. In addition, those of skill in the art will recognize appropriate variations from the procedures and reaction conditions specifically described herein, as well as substitutions for the specific chemical reagents and components that may be used, in accord with the practice of the present invention.

Formulation 1: Palmitoylpalmitoleoyl Phosphatidylcholine (16:0/16:1PC)

The use of this formulation is seen in situations e.g. where the establishment of a first or new pulmonary surfactant film is needed. By way of example, representative situations might be the establishment of the first surfactant film directly after birth in premature babies with surfactant deficiency, in situations of altered surfactant function/quantity e.g. after meconium aspiration or pneumonia or after inhalation/aspiration of toxic substances.

A preparation intended for this use will include an enriched concentration of 16:0/16:1PC. In particular applications, the amount of 16:0/16:1PC will comprise at least 50% or more, and even up to 95% to 100%, of the total PC content in the formulation.

Formulation 2: Palmitoylmyristoyl Phosphatidylcholine (16:0/14:0PC)

The use of this formulation is seen in situations e.g. where regular formation of the alveoli is inhibited or the architecture of healthy alveoli is disturbed. Hence, the particular architectural features of the pathological lung needs a particular fractional composition of the different PC species discussed herein, in particular an enhanced concentration of palmitoylmyristoyl-PC (16:0/14:0PC). By way of example, representative uses and situations include use in premature babies with primary or secondary decreased alveolarization, BPD as an example. Furthermore all situations with secondary destruction of alveoli leading to enlarged distal gas exchange units, e.g. emphysema, may be treated using these formulations enriched for palmitoylmyristoyl-PC (16:0/14:0PC).

Compositions and Pharmaceutical Kits:

In another aspect, the invention provides a pharmaceutical kit comprising a first and a second container means, the first container means comprising a lung surfactant composition of PC, and in particular palmitoylmyristoyl-PC (16:0/14:0PC), according to the invention and the second container means comprising a dispersion medium for the lung surfactant composition. The lung surfactant composition may be in powder or particulate form. Any of the above individual or combination of PC formulations may be included in the kit first container means comprising the lung surfactant composition.

The PC-containing pharmaceutical compositions of the invention may be in powder or particulate form adapted to be dispersed in an aqueous medium before use. For example, the pharmaceutical compositions of PC of the kit may be in solid (e.g. powder, particles, granules, sachets, tablets, capsules etc.), semi-solid (gels, pastes etc.) or liquid (solutions, dispersions, suspensions, emulsions, mixtures etc) form and adapted for administration via e.g. the respiratory organs. A pharmaceutical composition in liquid form may be in the form of a dispersion comprising the lung surfactant composition and an electrolyte solution such as, e.g. a composition that is adapted to physiological conditions e.g. a physiologically acceptable solution.

The pharmaceutical composition surfactant or surfactant supplement of the kits or individually provided products may further comprise another therapeutically, prophylactically and/or diagnostically active substance.

The pharmaceutical kit according to the present invention may include instructions with recommendations for the time period during which the lung surfactant composition should be administered after dispersion in the dispersion medium.

### Example 3

### Characterization of Changing Phospholipid Profile During Gestation and Early Postpartum Development

The present example is provided to demonstrate the utility of the present invention using total PC content in a subject lung sample as a tool in identifying the pulmonary developmental stage and any abnormalities thereof in an animal during gestation and early life (less than 18 months postpartum). The characteristic phospholipid profile may be used to identify and diagnose pulmonary developmental abnormalities, and hence aid in the identification of a suitable treatment regimen for the subject animal.

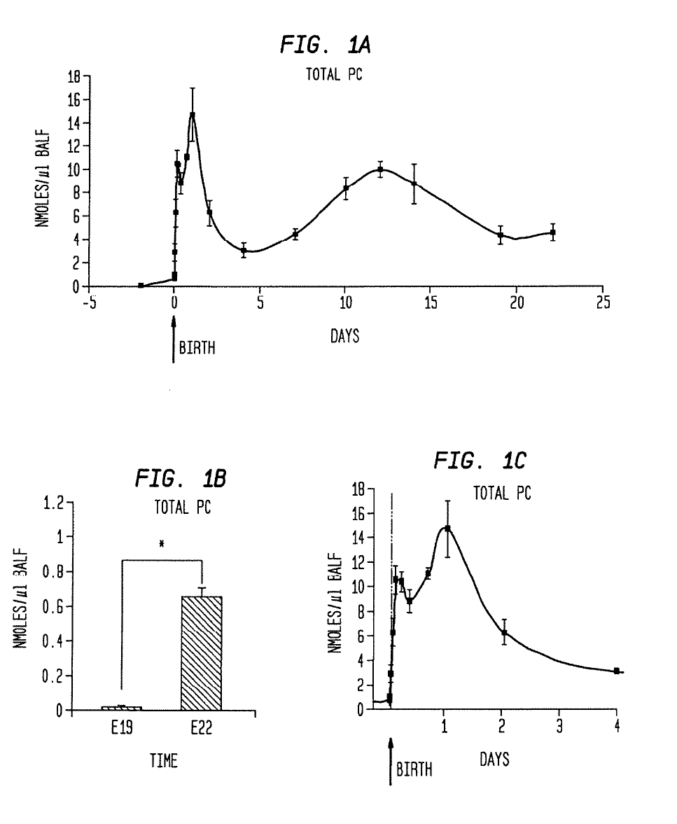

Bronchoalveolar lavage was performed on rats at differing gestational and postpartum ages. Total PC concentration was determined by the sum of the concentrations of all individual PC species. As can be seen in FIG. 1a, PC content in BALF varied tremendously during fetal and postnatal lung development. The amount of extracellular surfactant PC increased significantly (40-fold) between 19 and 22 days' gestation (FIG. 1b). A further 10-fold increase in total PC content of BALF occurred within the first two hours after birth (FIG. 1c).

This immediate rise in surfactant PC following birth may be attributed to two factors. Firstly, the decrease in alveolar fluid via clearance would concentrate the components of the bronchoalveolar compartment, including surfactant. At or shortly before birth the lung switches from a fluid secreting to a fluid absorbing organ. Although much of the lung fluid is cleared within 2 hours of birth there is evidence that the process of lung fluid adsorption is a more protracted process lasting more than 40 hours in the rat (23). Secondly, it is known that mechanical forces increase surfactant secretion (24-26). Lung expansion due to the first deep sigh or onset of breathing has been shown to stimulate the release of preformed lamellar bodies into the extracellular space (27, 28). The concentration of PC in BALF decreased slightly after 2 hours postpartum, but then increased and reached maximal levels at 24 hours after birth after which it declined and reached mature levels at day 4 postpartum (FIG. 1c). While not intending to be limited to any particular mechanism of action or theory, this second postnatal surge in PC content in BALF may at least in part be attendant the secretion of newly synthesized surfactant PC.

Earlier observations show that choline incorporation into rat lung tissue PC peaked on the first day after birth (see review (22)). The synthesis of new lamellar bodies has been estimated to require approximately 6 hours (29, 30). This may at least in part explain or be related to the phenomenon observed of a plateau between the two peaks at 2 and 24 hours postpartum. Thus, the requirement for surfactant during early extrauterine life is met by a release of preformed lamellar bodies within the first few hours of breathing, followed by massive synthesis and secretion by alveolar type II epithelial cells of new surfactant material.

Alternatively, the second peak could be a secretion from a more slowly released surfactant pool (31). How alveolar type II epithelial cells sense this need for new surfactant within 2 hours after the onset of breathing is unknown.

Another increase of total PC content in BALF occurred after postnatal day 4 with a peak at day 12 and a subsequent decline until day 19 when adult levels were reached (FIG. 1a). In rats, the bulk of alveolarization takes place between days 4 and 14 (14). The concomitant relationship between PC concentration and alveolarization identified in the present invention has never been reported.

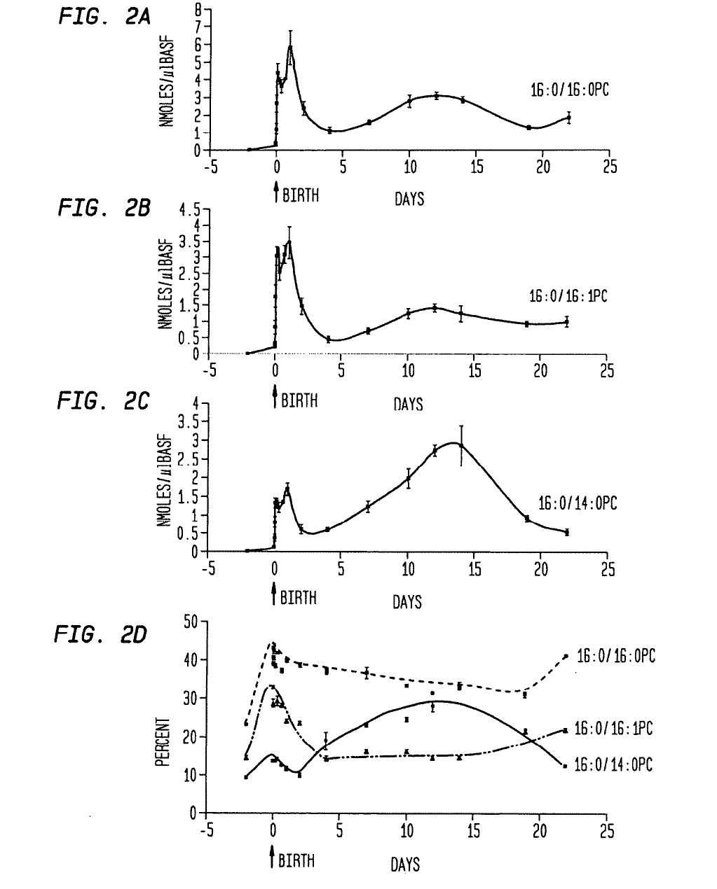

The enlargement in surface area that occurs during alveolarization would require an increasing amount of surfactant. The increase in the amount of surfactant PC during alveolarization suggests that there may be a co-regulation of surfactant and septal formation. Alveolar type II epithelial cell numbers are believed to peak during alveolarization (32). The increased number of type II cells could account for increased surfactant production during this time period. The increase in PC concentration during alveolarization is at least in part, and may be primarily due to, a phospholipid composition defined as palmitoylmyristoyl-PC (16:0/14:0PC) and not dipalmitoyl-PC (16:0/16:0PC) (FIG. 2d).

### Example 4

### Phopholipid During Fetal and Early Postnatal Development

The present example demonstrates the utility of the present invention for use as a surfactant replacement preparation or as part of a surfactant replacement therapy in the treatment of IRDS or other pulmonary function/developmental disorder, especially those related to prematurity and/or delayed/impaired alveolarization in infants.

The three predominant species of PC in surfactant (dipalmitoyl-PC (16:0/16:0PC), palmitoylpalmitoleoyl-PC (16:0/16:1PC) and palmitoylmyristoyl-PC (16:0/14:0PC) ) were examined by absolute concentration and percentage distribution (FIG. 2) in animals both before and after birth. Before birth, the proportion of dipalmitoyl-PC (16:0/16:0PC) increased from 23% at day 19 to 40% at birth (FIG. 2d; Table 1). The proportion of larger acyl chain unsaturated (16:0/18:1PC, 18:0/18:2PC, 16:0/20:4PC, 18:0/22:6PC and 18:1/18:2PC) PC declined (Table 1).

TABLE 1
Content of PC in Fetal Rat BALF

% Total PC

| PC Species | Day 19 | Day 22 |
|---|---|---|
| 16:0/14:0 | 9.0 +/− 0.4 | 13.2 +/− 0.3* |
| 16:0/16:1 | 14.1 +/− 0.5 | 33.0 +/− 0.3* |
| 16:0/16:0 | 22.9 +/− 0.4 | 38.8 +/− 0.5* |
| 16:0/18:2 | 9.6 +/− 0.7 | 2.8 +/− 0.1* |
| 16.0/18:1 | 24.5 +/− 0.9 | 8.6 +/− 0.3* |
| 18:1/18:2 | 4.0 +/− 0.3 | 1.8 +/− 0.2* |
| 18:0/18:2 | 3.8 +/− 0.1 | 0.5 +/− 0.0* |
| 18.0/20:4 | 2.7 +/− 0.2 | 0.6 +/− 0.1* |
| 18:0/22:6 | 5.1 +/− 0.3 | 0.4 +/− 0.0* |

*P ≦ 0.01. N = 4 separate BALF samples.

After birth the fractional concentration of dipalmitoyl-PC (16:0/16:0PC) remained consistently close to 40% (FIG. 2d; Table 1), although absolute values altered substantially (FIG. 2a). These data indicate that dipalmitoyl-PC(16:0/16:0PC) ratios with respect to total PC content is regulated to a near constant value (FIG. 2a). In some mammals, dipalmitoyl-PC (16:0/16:0PC) content in surfactant fluctuates between 35%-60% [rabbits: 35.6% (6); humans: 54% (6, 33)]. This may define an important concentration of 16:0/16:0PC for optimal surface-active function in vivo.

Surfactant from piglets is enriched in palmitoylpalmitoleoyl-PC (16:0/16:1PC) and palmitoylmyristoyl-PC (16:0/14:0PC) relative to adult pigs. The present data now demonstrates that these two PC species have a distinct profile during fetal and postnatal development (FIG. 2b, 2c, 2d). The fractional concentration of palmitoylpalmitoleoyl-PC (16:0/16:1PC) in BALF was greatest at birth (33%) and diminished postpartum (FIGS. 2b,2d; Table 1). There was no major change in its concentration between postnatal day 7 and 22 (FIG. 2d).

The high concentration of palmitoylpalmitoleoyl-PC (16:0/16:1PC) at birth (FIG. 2b) may aid in the establishment of the first air/liquid interface that is required at this time. This establishment requires a rapid adsorption of surfactant to the interface. Besides surfactant proteins B and C (35), palmitoyloleoyl-PC (16:0/18:1PC) has also been shown to improve dipalmitoyl-PC (16:0/16:0PC) adsorption at the air/liquid interface (11). Although palmitoyloleoyl-PC (16:0/18:1PC) may improve the adsorption rate of surfactant to the interface, it was found that its fractional concentration varied only little throughout development. Moreover, its concentration was far below that of palmitoylpalmitoleoyl-PC (16:0/16:1PC) around birth (33% for palmitoylpalmitoleoyl-PC (16:0/16:1PC) vs. 9% for palmitoyloleoyl-PC (16:0/18:1PC) ). Considering that palmitoylpalmitoleoyl-PC (16:0/16:1PC), like palmitoyloleoyl-PC (16:0/18:1PC), has a greater molecule to water ratio at the air/liquid interphase than dipalmitoyl-PC (16:0/16:0PC) at a given pressure, it may have better adsorption characteristics than dipalmitoyl-PC (16:0/16:0PC) (36-38). Therefore, palmitoylpalmitoleoyl-PC (16:0/16:1PC) is proposed to play an important role in forming the pulmonary surfactant film immediately after birth.

The palmitoylmyristoyl-PC (16:0/14:0PC) amount in BALF increased at birth, consistently with the rise in concentrations of dipalmitoyl-PC (16:0/16:0PC) and palmitoylpalmi toleoyl-PC (16:0/16:1PC) (FIG. 2c; Table 1). However, palrnitoylmyristoyl-PC (16:0/14:0PC) content in BALF peaked between days 12 to 14 postpartum (FIG. 2c). Fractional palmitoylmyristoyl-PC (16:0/14:0PC) concentrations were increased from postnatal days 7 to 14 (FIG. 2d), which corresponds to the alveolarization period in the rat (14). In fact, the general rise in total PC content during this time (FIG. 1aa) was primarily accounted for by the increase in palmitoylmyristoyl-PC (16:0/14:0PC) (FIG. 2d).

The function of palmitoylmyristoyl-PC (16:0/14:0PC) in pulmonary surfactant is unclear. It has a similar molecule to water ratio at the water/air interphase as dipalmitoyl phosphatidylcholine (16:0/16:0PC) at a given pressure (36, 37). The marginal chain asymmetry will likely not result in a pronounced difference over dipalmitoyl-PC (16:0/16:0PC) with respect to adsorption properties. Thus, it is unlikely that palmitoylmyristoyl-PC (16:0/14:0PC), in contrast to palmitoylpalmitoleoyl-PC (16:0/16:1PC) enhances the air/liquid adsorption rates of dipalmitoyl-PC (16:0/16:0PC). Considering the unclear role for palmitoylmyristoyl-PC (16:0/14:0PC) in surfactant function, additional work was done to determine whether the rise of palmitoylmyristoyl-PC (16:0/14:0PC) during alveolarization was specific to the rat. Therefore, BALF from mice was analyzed at late gestation (day 18) and around the peak (postnatal day 10) of alveolarization (15).

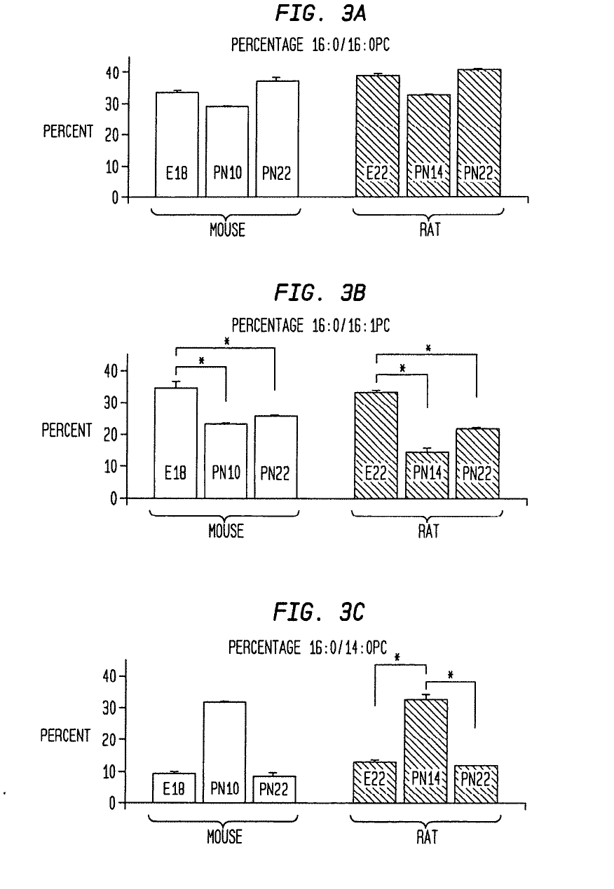

Mouse and rat BALF had similar fractional dipalmitoyl-PC (16:0/16:0PC) levels at fetal, postnatal (alveolar period) and mature time points (FIG. 3a). Likewise, there was a similar trend between the two animals for high palmitoylpalmitoleoyl-PC (16:0/16:1PC) content in fetal samples and high palmitoylmyristoyl-PC (16:0/14:0PC) content during alveolarization (FIG. 3b, 3ca). This consistency between the two rodent species suggests that the relatively high palmitoylpalmitoleoyl-PC (16:0/16:1PC) content of surfactant at birth as well as the relatively high palmitoylmyristoyl-PC (16:0/14:0PC) content of surfactant during alveolarization is a general phenomenon among all animals, and therefore has application and utility in the treatment of other animals, including humans.

### Example 5

### Surfactant Palmitoylmyristoyl Phosphatidylcholine (16:0/14:0PC) in Animal Models Having Compromised Alveolarization

The present example is presented to demonstrate the utility of the present invention as a marker useful in the detection of chemical and disease-related damage to the lung. The relative concentration of the PC species, palmitoylmyristoyl-PC (16:0/14:0PC) is demonstrated herein to be specifically correlated with the incidence of chemical-induced (such as dexamethasone) damage to alveolar tissue and/or reduced alveolarization in an animal. Palmitoylmyristoyl-PC (16:0/14:0PC) concentration is also demonstrated to be a reliable indicator of alveolar curvature and alveolar size in the animal lung.

Surfactant palmitoylmyristoyl-PC (16:0/14:0PC) increased during the period of alveolarization. To determine the nature of this relationship, BALF samples were examined from two rat models of diminished alveolarization. The two models employed were postnatal exposure to either dexamethasone or 60% oxygen. Postnatal administration of dexamethasone to rats has been described to result in a reduced alveolarization (17, 39-41). In the present study, a high dose, short-term treatment with dexamethasone was used to induce reduced alveolarization in the animal. After this treatment at the age of 14 days, a significant decrease in parenchymal complexity was observed in the treated animals, with larger and fewer lung alveoli compared to controls (17).

Neonatal hyperoxia of mice and rats may also be used to induce diminished alveolarization (18, 42-44). In the present studies, exposure of neonatal rats to 60% oxygen was observed to result in a significant reduction of total PC in BALF at postnatal day 10 (FIG. 4a). By postnatal day 14, total PC values were no longer significantly different between 60% oxygen- and 60% O₂-exposed animals (FIG. 4e).

Similar observations of reduced surfactant PC have been reported for neonatal rabbits exposed to 98% oxygen (45), although this effect could be the result of acute cellular injury. The content of individual PC molecules, including dipalmitoyl-PC (16:0/16:0PC), palmitoylmyristoyl-PC (16:0/14:0PC) and palmitoylpalmitoleoyl-PC (16:0/16:1PC), were also reduced in BALF of rats exposed to 60% oxygen (FIG. 4b-4h).

The overall reduction in surfactant PC synthesis may be due to several factors. Hyperoxia is known to induce the release of a number of cytokines (46). Some of these cytokines, in particular TNFα, have been shown to reduce the activity of the rate limiting enzyme in PC synthesis, CTP: phosphocholine cytidylyltransferase (47-50). In addition, oxidant stress has been shown to affect another crucial lipid synthesizing enzyme, glycerol phosphate acyltransferase (51). Alternatively, hyperoxia increases lipid peroxidation in rat alveolar type II epithelial cells (52). Therefore, alveolar type II epithelial cells may be shunting their lipid production from surfactant synthesis to cellular membrane repair.

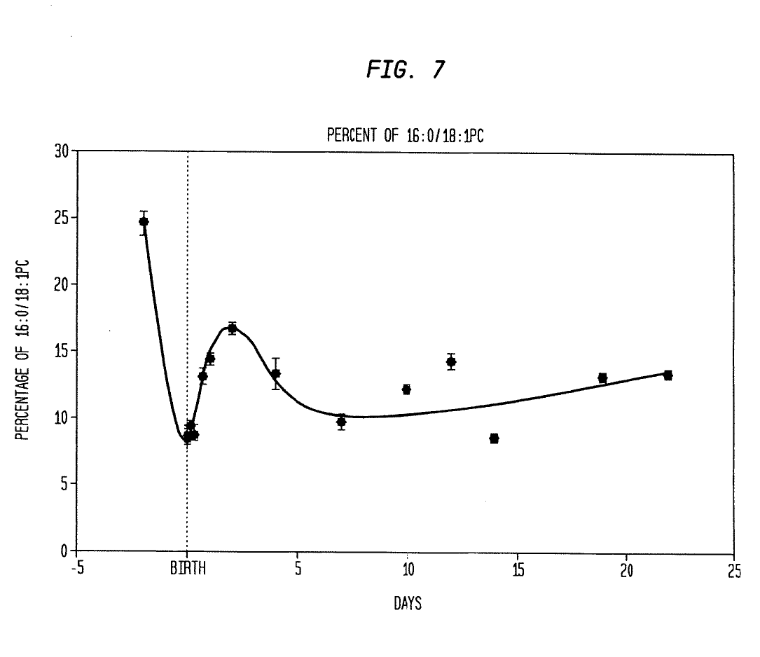

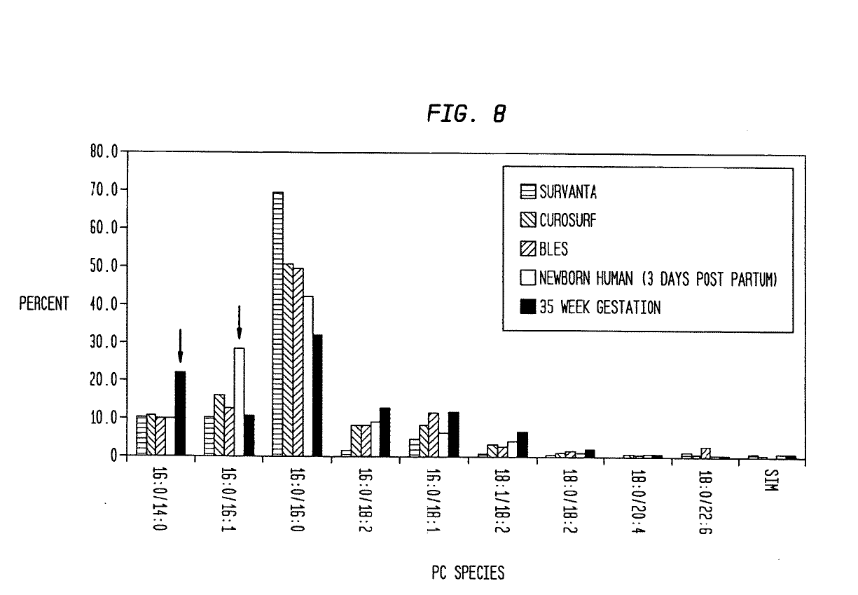

In contrast to hyperoxia, the present studies have demonstrated that post partum dexamethasone treatment results in a significant increase of total PC content in BALF at day 10 (FIG. 4a). In particular, monounsaturated PC molecules, such as palmitoylpalmitoleoyl-PC (16:0/16:1PC) (FIG. 4c) and palmitoyloleoyl-PC (16:0/18:1PC) (see FIG. 7) were elevated in BALF of dexamethasone-treated neonatal rats. Similar observations have been reported for liver PC of dexamethasone-treated rats (53). Saturated PC species, including dipalmitoyl-PC (16:0/16:0PC), were also elevated in BALF of dexamethasone-treated neonatal rats (FIG. 4b) with the exception of palmitoylmyristoyl-PC (16:0/14:0PC), which was significantly reduced (FIG. 4d). Palmitoylmyristoyl-PC (16:0/14:0PC) (FIG. 4h) was the only PC species (FIG. 4e-g) significantly reduced after 14 days of 60% oxygen exposure.

Thus, the two rat models of reduced alveolarization had contrasting effects on surfactant PC production, i.e. up regulation with dexamethasone and down regulation with 60% O₂. With respect to surfactant PC, a constant decrease was observed in palmitoylmyristoyl-PC (16:0/14:0PC) concentration. The results suggest that surfactant palmitoylmyristoyl-PC (16:0/14:0PC) concentrations relate to the alveolarization process not only during normal lung development, but also in two different models of diminished alveolar formation.

### Example 6

### Surfactant Palmitoylmyristoyl Phosphatidylcholine (16:0/14:0PC) Enriched Preparations and Pulmonary Disease

The present example is provided to demonstrate the utility of the present invention for use in detecting and/or diagnosing reduced pulmonary function and pulmonary disease in an animal, and particularly lung and/or alveolar related pathologies such as emphysema and bronchopulmonary dysplasia (BPD).

To evaluate the three predominant species of PC in human surfactant, tracheal aspirates were obtained from intubated neonates and BALF from adult humans. Data was subdivided based on pathology. Tracheal aspirates were obtained from infants with respiratory distress syndrome (RDS) who developed BPD and from those infants who did not develop BPD.

BALF was obtained from adult human patients with emphysema or idiopathic pulmonary fibrosis (IPF), and from post lung transplant patients with no pronounced alveolar complication. These samples were compared with BALF obtained from lung cancer patients that had normal alveolar architecture. Samples of emphysematic patients and infants who developed BPD displayed significantly reduced surfactant palmitoylmyristoyl-PC (16:0/14:0PC) levels compared to all other patient groups tested (FIG. 5b).

Dipalmitoyl-PC (16:0/16:0PC) did not significantly differ between the patient groups (FIG. 5a). Thus, the changes in palmitoylmyristoyl-PC (16:0/14:0PC) content in the emphysematic and BPD groups are likely not the result of a general loss of surfactant. Emphysema is characterized by abnormal, permanent enlargement of airspaces distal from the terminal bronchi, while one of the hallmarks of BPD seen in premature infants is alveolar simplification, i.e. larger but fewer alveoli. The reduced palmitoylmyristoyl-PC (16:0/14:0PC) content in lavage fluid of BPD and emphysematic patients is in line with the rat models of reduced alveolarization. As such, the results suggest that surfactant palmitoylmyristoyl-PC (16:0/14:0PC) content in humans also relate to distal airspace architecture of the lung.

From these studies, it is envisioned that a phospholipid composition comprising an enriched concentration of a surfactant palmitoylmyristoyl-PC (16:0/14:0PC) will provide a pharmaceutically effective lung surfactant therapy for patients having reduced alveolarization. Methods for treating an animal, such as a human, having been diagnosed as having reduced alveolarization, such as is characteristic of patients having emphysema, BPD, IRDS, and pathologies related thereto, are therefore provided by administering an effective amount of a pharmaceutically acceptable preparation of the surfactant palmitoylmyristoyl-PC(16:0/14:0PC) as described herein.

In addition, a method of the present invention is provided for identifying a patient having reduced alveolarization or other change in alveolar function/architecture related to disease, such as in emphysema and BPD. The method, for example, would comprise obtaining a lung tissue sample, such as a tracheal aspirate or biopsy, and measuring the amount of palmitoylmyristoyl-PC (16:0/14:0PC) in the sample, wherein a reduced concentration of palmitoylmyristoyl-PC (16:0/14:0PC) in the tissue sample compared to a control amount of palmitoylmyristoyl-PC (16:0/14:0PC) is diagnostic of reduced alveolar function or pulmonary disease in the animal.

### Example 7

### Palmitoylmyristoyl Phosphatidylcholine (16:0/14:0PC) During Alveolarization and as an Indicator of Alveolar Curvature

The present example is provided to demonstrate the utility of the invention as a pharmaceutically effective and specific lung surfactant preparation useful for enhancing alveolarization and lung development in an animal. The present study was performed on tissue obtained from rodents, and demonstrates the utility of the present preparations as useful in the treatment of other animals, including humans.

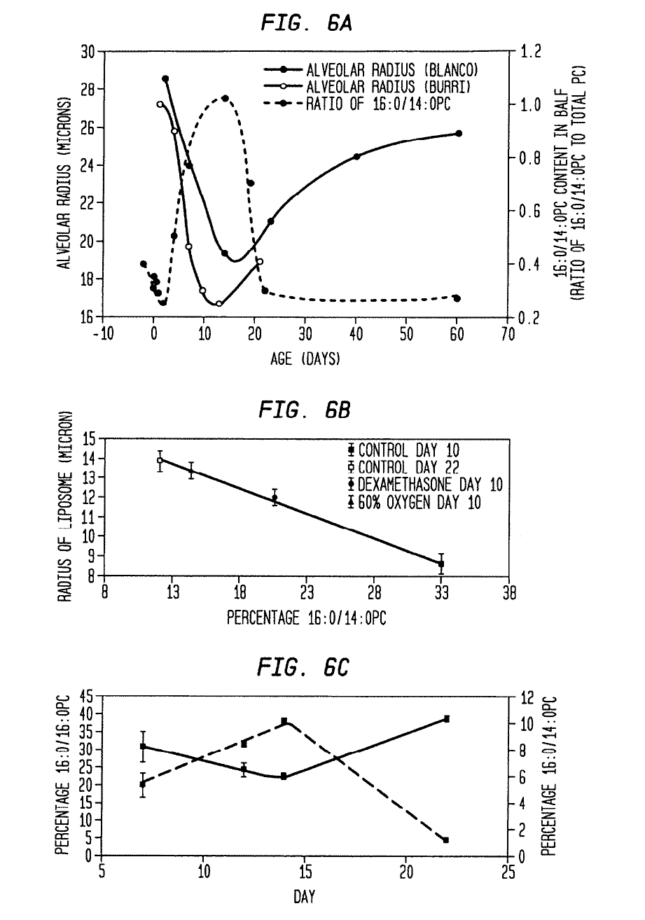

The main feature of alveolarization is the subdivision of the preexisting voluminous saccules by septation which leads to smaller units (alveoli) and an increase in total surface area. These units then have the potential to increase in size during growth of the animal. One of the implications of saccular subdivision is an increase in alveolar curvature. Two groups have reported alterations in alveolar diameter (14) and volume (54) during the period of alveolarization in the rat (14, 54, 55). When the calculated radii from the reported alveolar dimensions were plotted against palmitoylmyristoyl-PC (16:0/14:0PC) in rat BALF during rat development, a striking relationship between palmitoylmyristoyl-PC (16:0/14:0PC) content and alveolar curvature was identified by the present inventors (FIG. 6a).

To even further characterize this relationship, light scattering analysis was performed on liposomes formed from total lipids extracted from BALF. Lipids in BALF of day 10 neonatal rats, which have a high surfactant palmitoylmyristoyl-PC (16:0/14:0PC) content (FIG. 4d), formed liposomes with an average particle size of 9.0±0.4 microns. In contrast, lipids in BALF from dexamethasone and 60% oxygen-treated rats, which have a lower surfactant palmitoylmyristoyl-PC (16:0/14:0PC) content (FIG. 4d), formed significantly larger liposomes (13.2±0.7 microns and 12.6±0.6 microns, respectively). The BALF liposomes of 22 day old rats (14±0.7 micron) were significantly larger than those of 10 day-old rats consistent with the lower palmitoylmyristoyl-PC (16:0/14:0PC) content (FIG. 2cd). When the radii of BALF liposomes were plotted against BALF palmitoylmyristoyl-PC (16:0/14:0PC) content, a strong correlation (r₂=0.998) was found (FIG. 6b). The palmitoylmyristoyl-PC (16:0/14:0PC) therefore serves to at least in part increase the curving capacity of surfactant lipids. This would explain a potential basis for the increased palmitoylmyristoyl-PC (16:0/14:0PC) levels in surfactant of higher curved (smaller) air spaces that occurs during alveolarization. Other changes in variables such as surfactant proteins B and C may also be involved.

Intrinsic curvature in membranes can be influenced by small polar head lipids (56) or by asymmetric acyl chain length (57-59). Freeze fracture analysis of PC has shown that dipalmitoyl-PC (16:0/16:0PC) liposomes have a three-fold greater radius compared to palmitoylmyristoyl phosphatidylcholine (16:0/14:0PC) liposomes (60). The acyl chains of palmitoylmyristoyl-PC (16:0/14:0PC) will not have the same surface packing as dipalmitoyl-PC (16:0/16:0PC) at 37° C. However, because of its distinct acyl packing characteristics, it may obtain high surface pressures when spread on a highly curved interface. Therefore, palmitoylmyristoyl-PC (16:0/14:0PC) improves surfactant function during secondary septation, which is associated with more curved surface areas.

### Example 8

### Alveolar Type II Epithelial Cells and Palmitoylmyristoyl Phosphatidylcholine (16:0/14:0PC) Secretion During Lung Alveolarization

The present example is provided to demonstrate the correlation between alveolar Type II epithelial cell secretion of phosopholipid and early postnatal lung development.

Alveolar type II epithelial cells were found to play a role in controlling the acyl composition of PC during alveolarization. Using laser capture microscopy and mass spectral lipid analysis, palmitoylmyristoyl-PC (16:0/14:0PC) content of rat alveolar type II epithelial cells was found to increase postnatally from day 7; to peak at days 12-14 and to subsequently decline until day 21 when adult levels were reached (FIG. 6c). This profile for the increase and decrease in palmitoylmyristoyl-PC (16:0/14:0PC) content was also observed in bronchoalveolar lavage samples examined from animals at the same corresponding developmental time periods.

The similarity in the palmitoylmyristoyl-PC (16:0/14:0PC) content profiles from both the cellular (alveolar Type II epithelial cells) and bronchoalveolar lavage (BALF) samples during the alveolarization period demonstrates that lipid changes in the BALF are due to alveolar type II epithelial cells producing a different surfactant. How the alveolar type II epithelial cells sense architectural changes and produce acyl-specific PC during alveolarization remains to be elucidated.

### Example 9

### Palmitoylmyristoyl Phosphatidylcholine (16:0/14:0PC) Enriched Preparations for Treatment of Lung (Alveolar) Damage associated with Chemical Exposure

The present example demonstrates the utility of the invention in the treatment of pulmonary disease or damage resulting from pulmonary exposure to potentially toxic or otherwise damaging agents. It is envisioned that chronic exposure to commonly used inhalable preparations of steroids, such as those used in the treatment of asthma, results in alveolar/pulmonary damage that may be detected and treated using the surfactants, surfactant supplements, and pulmonary disease markers of the present invention.

The method for the prevention or treatment of pulmonary disease or destruction consequent exposure to chemical and steroidal elements is provided comprising introducing a phosopholipid preparation enriched for a phospholipid palmitoylmyristoyl-PC (16:0/14:0PC), alone or in combination with other active ingredients (such as Protein B, C, D, or other protein), in an amount effective to reduce the symptoms of or prevent pulmonary disease, wherein the pulmonary disease is reactive oxygen-induced or mediated pulmonary damage, chemically induced lung injury, injury due to oxygen radicals, injury due to ozone, injury due to chemotherapeutic agents, inflammatory and infectious diseases, reperfusion injury, drowning, lung transplantation, and organ (lung) rejection.

### Example 10

### Preparations having Enriched 16:0/14:0PC Concentrations with Pulmonary Surfactant Proteins

The surfactant formulations of the present invention comprise in some embodiments an enriched concentration of the PC, palmitoylmyristoyl-PC(16:0/14:0PC). These preparations may be formulated together with one or more important pulmonary surfactant proteins. Such pulmonary surfactant proteins include, by way of example, Pulmonary Surfactant protein A (SP-A), Pulmonary Surfactant Protein B (SP-B), Pulmonary Surfactant Protein C (SP-C) and Pulmonary Surfactant Protein D (SP-D).

These preparations may take the form of a lung “wash” (for use as a lavage), or may be formulated in an aerosol.

Combination with Pulmonary Surfactant Protein B (SP-B)—

It is envisioned that the palmitoylmyristoyl-PC (16:0/14:0PC)-enriched preparations of the present invention may be formulated to include an effective amount of the naturally occurring human surfactant Protein B, or a fragment thereof (e.g., N-terminal end fragment).

Naturally occurring SP-B has a length of 78 amino acid residues, an N-terminal residue of phenylalanine and a simple molecular weight of about 8,700. SP-B isolated from human lung migrates on polyacrylamide gels as an entity having a relative molecular weight (Mr) of 7-8,000 after sulfhydryl reduction. Without sulfhydryl reduction, the naturally occurring protein is also found as large oligomers. SP-B is hydrophobic, which is consistent with its in vivo strong association with phospholipids and solubility in organic solvents such as chloroform and methanol.

A porcine-derived Surfactant Protein B (about 0.2 mg/ml (0.2-0.4 mg/ml), extracted from porcine lungs, has been described in combination with DPPC (31 mg/ml), to form an intratracheal suspension, CUROSURF®. The formulations of the present invention in some embodiments are not envisioned to include porcine-derived SP-B. The present formulations would include an enriched concentration of 16:0/14:0PC, and similar concentrations of synthetic SP-B or porcine-derived SP-B may also be included.

Synthetic forms of Pulmonary Surfactant Protein B have been described in U.S. Pat. No. 6,660,833, U.S. Pat. No. 6,838,428 and U.S. Pat. No. 5,547,937. One example of a Protein-B based pulmonary preparation is Lucinactant.

A particular synthetic form of human Protein B is known as KL4. KL4 (also known as sinapultide) mimics the attributes of human SP-B. Native SP-B in natural pulmonary surfactant functions in surface tension lowering and promoting oxygen exchange. Chemically, KL4 consists of 21 amino acid residues where “K” is the amino acid lysine and “L” is the amino acid leucine. KL4-surfactant is an aqueous suspension consisting of KL4, the lipids DPPC and palmitoyloleoyl phosphatidylglycerol (POPG), plus the fatty acid, palmitic acid (PA).

Combination with Pulmonary Surfactant Protein C (SP-C)

It is envisioned that the palmitoylmyristoyl-PC (16:0/14:0PC)-enriched preparations of the present invention may be formulated to include an amount of the naturally occurring human surfactant Protein C, or a fragment thereof.

A synthetic (recombinant) Surfactant Protein C is described in U.S. Pat. No. 5,876,970. Native SP-C has an amino terminal glycine residue, a molecular weight of about 3,700, a polyvaline sequence, and is extremely hydrophobic. It is also substantially resistant to enzyme degradation by proteases (trypsin, chymotrypsin and staphylococcus nucleotide V-8), endoglycosidase F, and collagenase.

A calf-derived phospholipid preparation, INFASURF® (calfactant), has also been described. This preparation contains a natural surfactant from calf lungs including phospholipids (35 mg total phospholipids, including 26 mg phosphatidylcholine, of which 16 mg is disaturated phosphatidylcholine), neutral lipids, and hydrophobic surfactant-associated protein-B and C (SP-B and SP-C, 0.65 mg protein, including 0.26 mg of SP-B).

The formulations of the present invention in some embodiments may include calf-derived phospholipids, or may be formulated to primarily include synthetic, non-animal derived phospholipids. In some embodiments, the present inventive formulations would include an enriched concentration of palmitoylmyristoyl-PC (16:0/14:0PC), with or without synthetic SP-B and SP-C.

A bovine lung tissue extract prepared from minced calf lungs, has also been described that contains bovine phospholipids. One such preparation is SURVANTA®. The formulations of the present invention in some embodiments are envisioned to include bovine-lung derived phospholipids, or to instead include non-bovine lung, synthetic phospholipids. In some embodiments, the present inventive formulations would include an enriched (at least 50% by weight or more total phospholipid) concentration of palmitoylmyristoyl-PC (16:0/14:0PC).

Combination with Pulmonary Surfactant Protein D (SP-D)

It is envisioned that the palmitoylmyristoyl-PC (16:0/14:0PC)-enriched preparations of the present invention may be formulated to include an effective amount of the naturally occurring human surfactant Protein D, or a fragment thereof.

A synthetic (recombinant) Surfactant Protein D is described in U.S. Pat. No. 6,838,428. Native SP-D is a 43-kDa member of the collectin family of collagenous lectin domain—containing proteins that are expressed in epithelial cells of the lung. Synthetic forms of SP-D are described in Lu et al., Purification, “Characterization and cDNA cloning of Human Lung Surfactant Protein D”, (1992), Biochem. J. 284: 795-802, which is specifically incorporated herein by reference. Protein D is also described in U.S. Pat. No. 6,838,428, the teachings of which are also specifically incorporated herein by reference.

SP-D is associated with anti-pulmonary viral activity, and therefore is envisioned as particularly suitable for use in the compositions of the present phospholipid PC preparations to be administered to animals afflicted with some form of pulmonary viral disease, such as emphysema.

Combination with Colfosceril Palmitate (DPPC)

It is envisioned that the palmitoylmyristoyl-PC (16:0/14:0PC)-enriched preparations of the present invention may be formulated to include an amount of the phospholipid, colfosceril palmitate (commonly known as DPPC).

Colfoscheril palmitate has been included as a major component of preparations suitable as intratracheal suspensions that are protein-free. One such preparation formulated for use in infants is EXOSURF NEONATAL®. In suspension, EXOSURF NEONATAL® includes 13.5 mg/ml colfosceril palmitate, 1.5 mg/ml alcohol, and 1 mg/ml tyloxapol in 0.1 N NaCl. This preparation suspension is typically given directly to the lung through a tube (endotracheal administration).

It is envisioned that the palmitoylmyristoyl-PC (16:0/14:0PC)-enriched (about 50% by weight or more) preparations of the present invention may be formulated to include an effective amount of other species of phospholipids, such as DPPC, for example.

### Example 11

### Preparations having Enriched 16:0/14:0PC Concentrations

The present example defines the preparation as formulated from synthetic phospholipid sources.

The synthetic surfactant preparation for administration to premature infants includes about 30 mg/ml phosphatidylcholines comprising about 30% (12 mg/ml) palmitoylpalmitoleoyl-PC (16:0/16:1PC) and about 50% (15 mg/ml) dipalmitoyl-PC (16:0/16:0PC), about 20% (6 mg/ml) palmitoylmyristoyl-PC (16:0/14:0PC), about 0.2 to about 0.4 mg/ml synthetic SP-B, and about 0.5 mg synthetic SP-D, or is a natural adult (porcine or calf) surfactant enriched with palmitoylpalmitoleoyl-PC (16:0/16:1PC) and palmitoylmyristoyl-PC (16:0/14:0PC) to an end concentration of at least about 30% and about 20%, respectively (see FIG. 8).

The synthetic surfactant preparation for administration to emphysematic patients, in some embodiments, includes about 30 mg/ml phosphatidylcholines comprising about 20% (6 mg/ml) palmitoylpalmitoleoyl-PC (16:0/16:1PC), about 50% (15 mg/ml) dipalmitoyl-PC (16:0/16:0PC), about 30% (9 mg/ml) palmitoylmyristoyl-PC (16:0/14:0PC), about 0.2 to about 0.4 mg/ml synthetic SP-B and about 0.5 mg/ml synthetic SP-D. In other embodiments, the synthetic surfactant preparation comprises a natural adult (porcine or calf) surfactant enriched with palmitoylpalmitoleoyl-PC (16:0/16:1PC) (at least about 20%) and palmitoylmyristoyl-PC (16:0/14:0PC) (at least about 30%), and about 0.5 mg/ml synthetic SP-D.

### Example 12

### b 16:0/14:0PC as a Marker to Predict Risk in Infants for Continued Respiratory Distress

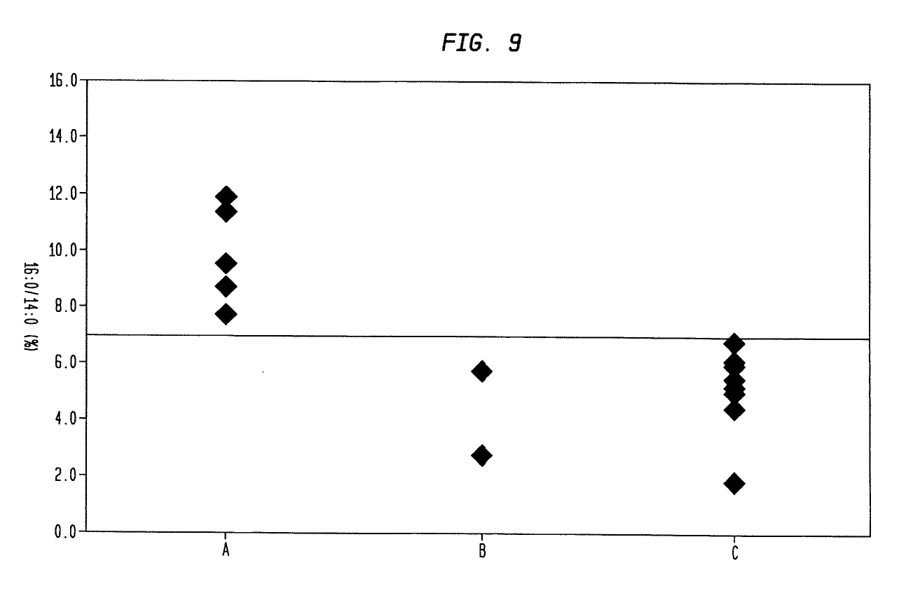

The present example is provided to demonstrate the utility of the invention as a method for predicting risk in a premature infant with respiratory distress for future development of bronchopulmonary dysplasia. By employing palmitoylmyristoyl-PC (16:0/14:0PC) as a marker in samples from premature infants with respiratory distress, one can effectively predict with a high degree of accuracy the percentage of these infants that will continue to suffer from pulmonary compromised conditions (see FIG. 9).

As shown in FIG. 9, a significant number of human infants diagnosed with RDS that went on to develop BPD (CLD) had an 16:0/14:0PC content (%) of 7% or less. Human infant samples taken from infants that had RDS and had already been diagnosed with BPD also had a 16:0/14:0PC content (%) of 7% or less. In contrast, samples obtained from human infants diagnosed with RDS that did not develop BPD (CLD) had a 16:0/14:0PC content (%) of more than about 7%. Hence, a reduced relative concentration percent (%) of 16:0/14:0PC (such as for example, about 10%, 20%, 25% or as much as 40% less 16:0/14:0PC compared to a control infant lung sample) in an infant lung sample is a prognostic indicator for identifying an infant at risk for developing BPD (CLD).

All documents, patents, journal articles and other materials cited in the present application are hereby incorporated by reference.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart there from.

### BIBLIOGRAPHY

The following references are specifically incorporated herein by reference.
1. Goerke J. Pulmonary surfactant: functions and molecular composition. Biochim Biophys Acta 1998; 1408(2-3):79-89.
2. Whitsett J A, and Weaver T E. Hydrophobic surfactant proteins in lung function and disease. N Engl J Med 2002; 347(26):2141-8.
3. Adams F H, Fujiwara T, Emmanouilides G C, and Raiha N. Lung phospholipids of human fetuses and infants with and without hyaline membrane disease. J Pediatr 1970; 77(5):833-41.
4. Bernhard W, Gebert A, Vieten G, Rau G A, Hohlfeld J M, Postle A D, and Freihorst J. Pulmonary surfactant in birds: coping with surface tension in a tubular lung. Am J Physiol Regul Integr Comp Physiol 2001; 281(1):R327-37.
5. Bernhard W, Haagsman H P, Tschernig T, Poets C F, Postle A D, van Eijk M E, and von der Hardt H. Conductive airway surfactant: surface-tension function, biochemical composition, and possible alveolar origin. Am J Respir Cell Mol Biol 1997; 17(1):41-50.
6. Postle A D, Heeley E L, and Wilton D C. A comparison of the molecular species compositions of mammalian lung surfactant phospholipids. Comp Biochem Physiol A Mol Integr Physiol 2001; 129(1):65-73.
7. Bernhard W, Hoffmann S, Dombrowsky H, Rau G A, Kamlage A, Kappler M, Haitsma J, Freihorst J, von der Hardt H, and Poets C F. Phosphatidylcholine molecular species in lung surfactant: composition in relation to respiratory rate and lung development. Am J Respir Cell Mol Biol 2001; 25(6):725-31.
8. Notter R H, Shapiro D L, Ohning B, and Whitsett J A. Biophysical activity of synthetic phospholipids combined with purified lung surfactant 6000 dalton apoprotein. Chem Phys Lipids 1987; 44(1):1-17.
9. Notter R H, Finkelstein J N, and Taubold R D. Comparative adsorption of natural lung surfactant, extracted phospholipids, and artificial phospholipid mixtures to the air-water interface. Chem Phys Lipids 1983; 33(1):67-80.
10. Fleming B D, and Keough K M. Surface respreading after collapse of monolayers containing major lipids of pulmonary surfactant. Chem Phys Lipids 1988; 49(1-2):81-6.
11. Holm B A, Wang Z, Egan E A, and Notter R H. Content of dipalmitoyl phosphatidylcholine in lung surfactant: ramifications for surface activity. Pediatr Res 1996; 39(5):805-11.
12. O'Brodovich H M. Immature epithelial Na+channel expression is one of the pathogenetic mechanisms leading to human neonatal respiratory distress syndrome. Proc Assoc Am Physicians 1996; 108(5):345-55.
13. Avery M E. Prevention of hyaline membrane disease. Pediatrics 1972; 50(4):513-4.
14. Burri P H, Dbaly J, and Weibel E R. The postnatal growth of the rat lung. I. Morphometry. Anat Rec 1974; 178(4):711-30.
15. Amy R W, Bowes D, Burri P H, Haines J, and Thurlbeck W M. Postnatal growth of the mouse lung. J Anat 1977; 124(1):131-51.
16. Burri P H. 1999. Lung development and pulmonary angiogenesis. Oxford University Press, New York.
17. Schwyter M, Burri P H, and Tschanz S A. Geometric properties of the lung parenchyma after postnatal glucocorticoid treatment in rats. Biol Neonate 2003; 83(1):57-64.
18. Han R N, Buch S, Tseu I, Young J, Christie N A, Frndova H, Lye S J, Post M, and Tanswell A K. Changes in structure, mechanics, and insulin-like growth factor-related gene expression in the lungs of newborn rats exposed to air or 60% oxygen. Pediatr Res 1996; 39(6):921-9.
19. Bligh E G, Dryer, W. J. A rapid method of total lipid extraction and purification. Can J Biochem physiol 1959; 37:911-917.
20. Ridsdale R, Tseu I, Roth-Kleiner M, Wang J, and Post M. Increased phosphatidylcholine production but disrupted glycogen metabolism in fetal type II cells of mice that overexpress CTP: Phosphocholine cytidylyltransferase. J Biol Chem 2004; 279(53):55946-57.
21. de la Maza A, Coderch L, Lopez O, and Parra J L. Transmission electron microscopy and light scattering studies on the interaction of a nonionic/anionic surfactant mixture with phosphatidylcholine liposomes. Microsc Res Tech 1998; 40(1):63-71.
22. Cockshutt A M, and Possmayer F. Metabolism of Surfactant Lipids and Proteins in the Developing Lung. In: Robertson B, Van Golde L M, Batenburg J J, editors. Pulmonary Surfactant From Molecular Biology to Clinical Practice. Amsterdam, The Netherlands: Elsevier Science Publishers; 1992. p. 339-377.
23. Folkesson H G, Matthay M A, Chapin C J, Porta N F, and Kitterman J A. Distal air space epithelial fluid clearance in near-term rat fetuses is fast and requires endogenous catecholamines. Am J Physiol Lung Cell Mol Physiol 2002; 282(3):L508-15.
24. U.S. Pat. No. 6,838,428—Whitsett, et al.
25. Thet L A, Clerch L, Massaro G D, and Massaro D. Changes in sedimentation of surfactant in ventilated excised rat lungs. Physical alterations in surfactant associated with the development and reversal of atelectasis. J Clin Invest 1979; 64(2):600-8.
26. Wirtz H R, and Dobbs L G. Calcium mobilization and exocytosis after one mechanical stretch of lung epithelial cells. Science 1990; 250(4985):1266-9.
27. Lawson E E, Birdwell R L, Huang P S, and Taeusch H W, Jr. Augmentation of pulmonary surfactant secretion by lung expansion at birth. Pediatr Res 1979; 13(5 Pt 1):611-4.
28. Nijjar M S. Ventilation-induced release of phosphatidylcholine from neonatal-rat lungs in vitro. Biochem J 1984; 221(3):577-86.
29. Baritussio A G, Magoon M W, Goerke J, and Clements J A. Precursor-product relationship between rabbit type II cell lamellar bodies and alveolar surface-active material. Surfactant turnover time. Biochim Biophys Acta 1981; 666(3):382-93.
30. Bernhard W, Pynn C J, Jaworski A, Rau G A, Hohlfeld J M, Freihorst J, Poets C F, Stoll D, and Postle A D. Mass spectrometric analysis of surfactant metabolism in human volunteers using deuteriated choline. Am J Respir Crit Care Med 2004; 170(1):54-8.
31. Dietl P, Frick M, Mair N, Bertocchi C, and Haller T. Pulmonary consequences of a deep breath revisited. Biol Neonate 2004; 85(4):299-304.
32. Kauffman S L, Burri P H, and Weibel E R. The postnatal growth of the rat lung. II. Autoradiography. Anat Rec 1974; 180(1):63-76.
33. Bernhard W, Mottaghian J, Gebert A, Rau G A, von Der H H, and Poets C F. Commercial versus native surfactants. Surface activity, molecular components, and the effect of calcium. Am J Respir Crit Care Med 2000; 162(4 Pt 1):1524-33.
34. Rau G A, Vieten G, Haitsma J J, Freihorst J, Poets C, Ure B M, and Bernhard W. Surfactant in newborn compared with adolescent pigs: adaptation to neonatal respiration. Am J Respir Cell Mol Biol 2004; 30(5):694-701.
35. Schram V, Anyan W R, and Hall S B. Non-cooperative effects of lung surfactant proteins on early adsorption to an air/water interface. Biochim Biophys Acta 2003; 1616(2):165-73.
36. Evans R W, Williams M A, and Tinoco J. Surface areas of 1-palmitoyl phosphatidylcholines and their interactions with cholesterol. Biochem J 1987; 245(2):455-62.
37. Seelig J, Dijkman R, and de Haas G H. Thermodynamic and conformational studies on sn-2-phosphatidylcholines in monolayers and bilayers. Biochemistry 1980; 19(10):2215-9.
38. Smaby J M, and Brockman H L. Surface dipole moments of lipids at the argon-water interface. Similarities among glycerol-ester-based lipids. Biophys J 1990; 58(1):195-204.
39. Massaro D, Teich N, Maxwell S, Massaro G D, and Whitney P. Postnatal development of alveoli. Regulation and evidence for a critical period in rats. J Clin Invest 1985; 76(4):1297-305.
40. Sahebjami H, and Domino M. Effects of postnatal dexamethasone treatment on development of alveoli in adult rats. Exp Lung Res 1989; 15(6):961-73.
41. Srinivasan G, Bruce E N, Houtz P K, and Bruce M C. Dexamethasone-induced changes in lung function are not prevented by concomitant treatment with retinoic acid. Am J Physiol Lung Cell Mol Physiol 2002; 283(2):L275-87.
42. Warner B B, Stuart L A, Papes R A, and Wispe J R. Functional and pathological effects of prolonged hyperoxia in neonatal mice. Am J Physiol 1998; 275(1 Pt 1):L110-7.
43. Thibeault D W, Mabry S, and Rezaiekhaligh M. Neonatal pulmonary oxygen toxicity in the rat and lung changes with aging. Pediatr Pulmonol 1990; 9(2):96-108.
44. Yi M, Jankov R P, Belcastro R, Humes D, Copland I, Shek S, Sweezey N B, Post M, Albertine K H, Auten R L, and Tanswell A K. Opposing Effects of 60% Oxygen and Neutrophil Influx on Alveologenesis in the Neonatal Rat. Am J Respir Crit Care Med 2004; 170(11): 1188-96.
45. Issa A, Lappalainen U, Kleinman M, Bry K, and Hallman M. Inhaled nitric oxide decreases hyperoxia-induced surfactant abnormality in preterm rabbits. Pediatr Res 1999; 45(2):247-54.
46. Varughese R, Nayak J L, LoMonaco M, O'Reilly M A, Ryan R M, and D'Angio C T. Effects of hyperoxia on tumor necrosis factor alpha and Grobeta expression in newborn rabbit lungs. Lung 2003; 181(6):335-46.
47. Vivekananda J, Smith D, and King R J. Sphingomyelin metabolites inhibit sphingomyelin synthase and CTP:phosphocholine cytidylyltransferase. Am J Physiol Lung Cell Mol Physiol 2001; 281(1):L98-L107.
48. Carroll J L, Jr., McCoy D M, McGowan S E, Salome R G, Ryan A J, and Mallampalli R. K., Pulmonary-specific expression of tumor necrosis factor-alpha alters surfactant lipid metabolism. Am J Physiol Lung Cell Mol Physiol 2002; 282(4):L735-42.
49. Mallampalli R K, Ryan A J, Salome R G, and Jackowski S. Tumor necrosis factor-alpha inhibits expression of CTP:phosphocholine cytidylyltransferase. J Biol Chem 2000; 275(13):9699-708.
50. Salome R G, McCoy D M, Ryan A J, and Mallampalli R K. Effects of intratracheal instillation of TNF-alpha on surfactant metabolism. J Appl Physiol 2000; 88(1):10-6.
51. Haagsman H P, Schuurmans E A, Alink G M, Batenburg J J, and van Golde L M. Effects of ozone on phospholipid synthesis by alveolar type II cells isolated from adult rat lung. Exp Lung Res 1985; 9(1-2):67-84.
52. Luo X, Christie N A, McLaughlin M A, Belcastro R, Sedlackova L, Cabacungan J, Freeman B A, and Tanswell A K. H2O2 mediates O2 toxicity in cultured fetal rat distal lung epithelial cells. Free Radic Biol Med 1999; 26(11-12):1357-68.
53. Brenner R R, Ayala S, and Garda H A. Effect of dexamethasone on the fatty acid composition of total liver microsomal lipids and phosphatidylcholine molecular species. Lipids 2001; 36(12):1337-45.
54. Blanco L N, Massaro G D, and Massaro D. Alveolar dimensions and number: developmental and hormonal regulation. Am J Physiol 1989; 257(4 Pt 1):L240-7.
55. Blanco L N, Massaro D, and Massaro G D. Alveolar size, number, and surface area: developmentally dependent response to 13% O2. Am J Physiol 1991; 261(6 Pt 1):L370-7.
56. Leikin S, Kozlov M M, Fuller N L, and Rand R P. Measured effects of diacylglycerol on structural and elastic properties of phospholipid membranes. Biophys J 1996; 71(5):2623-32.
57. Fuller N, and Rand R P. The influence of lysolipids on the spontaneous curvature and bending elasticity of phospholipid membranes. Biophys J 2001; 81(1):243-54.
58. Tate M W, and Gruner S M. Lipid polymorphism of mixtures of dioleoylphosphatidylethanolamine and saturated and monounsaturated phosphatidylcholines of various chain lengths. Biochemistry 1987; 26(1):231-6.
59. Szule J A, Fuller N L, and Rand R P. The effects of acyl chain length and saturation of diacylglycerols and phosphatidylcholines on membrane monolayer curvature. Biophys J 2002; 83(2):977-84.
60. Meyer H W, Semmler K, Rettig W, Pohle W, Ulrich A S, Grage S, Selle C, and Quinn P. J. Hydration of DMPC and DPPC at 4 degrees C. produces a novel subgel phase with convexconcave bilayer curvatures. Chem Phys Lipids 2000; 105(2):149-66.
61. Remingtons Pharmaceutical Sciences, Mack Publishing Co., Easton, Pa.
62. Bernhard et al. Mass Spectrometric Analysis of Surfactant Metabolism in Human Volunteers using Deuteriated Choline, Am. J. Crit. Care Med. (2004), 170 (1): 54-8.
63. U.S. Pat. No. 5,260,284—Lichtenberger;
64. U.S. Pat. No. 6,129,934—Egan et al.
65. U.S. Pat. No. 6,660,883—Walther et al.
66. U.S. Pat. No. 6,770,619—Larsson et al.

What is claimed is:

1. A phospholipid preparation suitable for use as a lung surfactant or lung surfactant supplement comprising an enriched concentration of palmitoylmyristoyl-PC (16:0/14:0PC) in a pharmacologically acceptable carrier solution.

2. A marker of reduced alveolarization or pulmonary disease state in a subject animal comprising a subject animal sample palmitoylmyristoyl-PC (16:0/14:0PC) concentration.

3. The marker of claim 2 wherein a reduced alveolarization or pulmonary disease state in the subject animal is indicated by a reduced subject animal sample palmitoylmyristoyl-PC (16:0/14:0PC) concentration.

4. The marker of claim 3 wherein the subject animal lung sample palmitoylmyristoyl-PC (16:0/14:0PC) concentration is reduced about 20% relative to a control animal lung sample palmitoylmyristoyl-PC (16:0/14:0PC) concentration as determined by mass spectral analysis.

5. The marker of claim 4 wherein the subject animal lung sample palmitoylmyristoyl-PC (16:0/14:0PC) concentration is reduced about 25% relative to the control animal lung sample palmitoylmyristoyl-PC (16:0/14:0PC) concentration as determined by mass spectral analysis.

6. A method for treating an animal having a compromised pulmonary condition comprising administering a pharmacologically effective amount of a surfactant preparation having an enriched concentration of palmitoylmyristoyl-PC (16:0/14:0PC) relative to other phospholipids in a pharmacologically acceptable carrier.

7. The method of claim 6 wherein the surfactant preparation of palmitoylmyristoyl-PC (16:0/14:0PC) comprises an enriched concentration of more than 50% of the phospholipids of the preparation.

8. The method of claim 6 wherein the surfactant preparation is essentially free of phospholipids other than palmitoylmyristoyl-PC (16:0/14:0PC).

9. A method for identifying a subject having reduced alveolarization or other change in alveolar function/architecture related to disease or exposure to a toxic substance comprising;
obtaining a tissue lung sample from a subject animal to provide a subject lung sample;
measuring an amount of palmitoylmyristoyl-PC (16:0/14:0PC) in the subject lung sample; and
comparing the amount of palmitoylmyristoyl-PC (16:0/14:0PC) in the subject lung sample to an amount of palmitoylmyristoyl-PC (16:0/14:0PC) in a control lung sample obtained from a control animal,
wherein a reduced concentration of palmitoylmyristoyl-PC (16:0/14:0PC) in the subject lung sample compared to the amount of palmitoylmyristoyl-PC (16:0/14:0PC) in the control lung sample is diagnostic of reduced alveolar function or pulmonary disease in the subject animal.

10. The method of claim 9 wherein the reduced alveolar function related to disease is emphysema, respiratory distress syndrome, idiopathic pulmonary fibrosis, broncho pulmonary dysplasia or diseases with primary defects of alveolarizaion.

11. The method of claim 9 wherein the amount of palmitoylmyristoyl-PC (16:0/14:0PC) in the subject lung sample is reduced at least 20% compared to the amount of palmitoylmyristoyl-PC (16:0/14:0PC) in the control lung sample.

12. The method of claim 9 wherein the subject lung sample has an elevated amount of total phospholipids compared to the total amount of phospholipids of a control lung sample.

13. The method of claim 12 wherein the total phospholipids comprise palmitoylpalmitoleoyl-PC (16:0/16:1PC) and palmitoyloleoyl-PC (16:0/18.1PC).

14. The method of claim 9 wherein the subject lung sample has an elevated amount of dipalmitoyl-PC(16:0/16:0PC) relative to the amount of dipalmitoyl-PC (16:0/16:0PC) in the control lung sample.

15. The method of claim 14 wherein the subject lung sample has an elevated amount of dipalmitoyl-PC (16:0/16:0PC) of about 20% to about 40% greater than the amount of dipalmitoyl-PC(16:0/16:0PC) in the control lung sample.

16. The method of claim 14 wherein the subject lung sample has an elevated amount of dipalmitoyl-PC (16:0/16:0PC) of about 28% to about 33% greater than the amount of dipalmitoyl phosphatidylcholine (16:0/16:0PC) in the control lung sample.

17. The method of claim 9 wherein the subject lung sample has an elevated amount of palmitoylpalmitoleoyl-PC (16:0/16:1PC) compared to the amount of palmitoylpalmitoleoyl-PC (16:0/16:1PC) in the control lung sample.

18. A surfactant pretreatment suitable for inhibiting lung damage comprising a preparation enriched for palmitoylmyristoyl-PC (16:0/14:0PC).

19. The surfactant pretreatment of claim 18 suitable for reducing lung damage attendant lung-ischemia-reperfusion injury.

20. A method for reducing lung damage attendant lung ischemia-reperfusion in a patient having a lung transplant comprising pretreating the patient with the surfactant pretreatment of claim 18.
